(19) United States
(12) Patent Application Publication
(10) Pub. No.: US 2026/0000341 A1
(43) Pub. Date: Jan. 1, 2026

(54) MUSCLE PROBE, SYSTEM AND METHOD

(71) Applicant: THE UNIVERSITY OF SHEFFIELD, Sheffield South Yorkshire (GB)

(72) Inventors: James John Philip ALIX, Sheffield South Yorkshire (GB); John Charles Clifford DAY, Bristol Avon (GB)

(73) Assignee: THE UNIVERSITY OF SHEFFIELD, Sheffield South Yorkshire (GB)

(21) Appl. No.: 19/113,888

(22) PCT Filed: Sep. 25, 2023
(86) PCT No.: PCT/EP2023/076380

§ 371 Date: Mar. 20, 2025

(30) Foreign Application Priority Data
Sep. 27, 2022 (GB) 2214072.7

(51) Int. Cl. A61B 5/397; A61B 5/00; A61B 5/262; A61B 5/296; A61B 5/395

(52) U.S. Cl. CPC A61B 5/397; A61B 5/0062; A61B 5/0071; A61B 5/0075; A61B 5/0084; A61B 5/262; A61B 5/296; A61B 5/395; A61B 5/4848; A61B 5/6848; A61B 5/7271; A61B 2560/0468

(57) ABSTRACT

A muscle probe is provided for obtaining electromyography data and optical spectroscopy data from muscle tissue. The muscle probe comprises an elongate needle having an outer wall surrounding a needle interior, the needle interior comprising: a core electromyography electrode; and one or more optical fibres; wherein the needle is arranged to be inserted into a muscle, and further arranged to detect electrical activity from the muscle; and wherein the one or more optical fibres are arranged to direct incident light from a light source toward a target area of the muscle, and further arranged to receive scattered light from the target area. The present disclosure aims to provide a muscle probe to improve the diagnostic pathway for patients with neuromuscular disorders, by developing a minimally invasive bedside test of muscle health.

### FIELD OF THE DISCLOSURE

The present disclosure relates to muscle probes, and in particular to muscle probes for obtaining electromyography and optical spectral data from muscle tissue.

### BACKGROUND TO THE DISCLOSURE

Neuromuscular disorders result in muscle weakness, cause significant morbidity and mortality and can take years to diagnose, often requiring invasive tests. They are in need of a better diagnostic pathway. Current key diagnostic tests include needle electromyography, muscle biopsy and genetic analyses.

Many of these conditions take a long time to diagnose and require invasive biopsies that are only undertaken in highly specialised centres. During this time opportunities for interventions and clinical trial participation are missed.

Electromyography examines the electrical activity arising from muscle. It has low specificity for individual conditions and its interpretation can be highly subjective. Electromyography is typically not used to monitor disease progression in neuromuscular disease, owing largely to its subjective nature, variability and limited quantitative outputs. As electromyography only studies the electrical activity of muscle it is ‘blind’ to other pathological changes, such as the infiltration of cells not usually present in muscle. Electromyography is also a specialist tool which must be implemented by skilled and experienced staff. This means higher costs and fewer available medical appointments.

While traditional muscle biopsy can be used to determine the presence of a neuromuscular disorder, only one muscle is typically biopsied. Areas of pathology in this muscle may be missed and other muscles might be better potential targets. Due to its invasive nature muscle biopsy is not typically used to monitor patients in clinical practice, and is only rarely used to monitor treatment response in clinical trials.

It is therefore desirable to provide a more precise, less invasive alternative to currently available techniques.

### SUMMARY OF THE DISCLOSURE

The present invention provides a combination of electromyography (EMG) and optical spectroscopy for improving the diagnostic pathway for patients with neuromuscular disorders, by developing a minimally invasive bedside test of muscle health. The facility of the invention for optical spectroscopy may preferably include Raman spectroscopy. This solution combines the multiple muscle testing of EMG with the molecular specificity typically available only from a traditional muscle biopsy—in this case using optical spectroscopy—but without the invasiveness and single muscle sampling typically encountered with such traditional muscle biopsy procedures. This combination also provides an improved biomarker of muscle health and/or disease, which may be used for diagnosing disease, monitoring disease progression, as a predictor of disease prognosis and/or treatment response.

In accordance with a first aspect of the present disclosure, there is provided a muscle probe comprising: an elongate needle having an outer wall surrounding a needle interior, the needle interior comprising: a core electromyography electrode; and one or more optical fibres; wherein the needle is arranged to be inserted into a muscle, and further arranged to detect electrical activity from the muscle; and wherein the one or more optical fibres are arranged to direct incident light from a light source toward a target area of the muscle, and further arranged to receive scattered light from the target area.

The needle is preferably intended to function as a typical electromyography needle as will be understood by the skilled addressee. In particular, the needle is preferably intended for use in detecting muscle membrane depolarisation, in which a potential difference is observed between the needle outer wall (acting as a “reference”) and the core (which acts as the active electrode).

The use of electromyography in the present probe includes confirmation that the probe is in muscle, in particular specific muscles that can be difficult to accurately target by surface palpation alone. It can also inform real-time guidance of the probe toward target areas of muscle which could include areas demonstrating electrical (or EMG) abnormalities, as well as areas of muscle which appear to be normal (as judged by an operator skilled in EMG). Once at a target area, an optical spectroscopic assessment may be performed using one or more optical fibres. Therefore, in preferable embodiments, the scattered light comprises inelastic scattered light, for assessment using optical spectroscopy. The inelastic scattered light preferably comprises one or more of: Raman scattered light; fluorescence scattered light; Brillouin scattered light. Said spectroscopic assessment preferably determines the molecular composition of the muscle for improved assessment of muscle health and/or neuromuscular disease status, and preferably provides a data fingerprint including a characterisation of said molecular composition. Said spectroscopic assessment can therefore be accurately guided to areas of interest within muscle, and possibly to specific muscles that would be difficult to accurately target by palpation alone. In addition, the probe could be used such that optical spectra were used to guide an EMG assessment.

The use of such a real-time EMG-guided biochemical assessment of a target muscle region preferably avoids the need for painful, slow and labour intensive traditional muscle biopsies. The improved specificity and precision of the present combination preferably reduces the time taken for an EMG assessment, while also reducing the number of muscles required to be examined, which preferably leads to reduced cost and improved patient comfort.

Determining muscle health and/or disease state, together with prognosis and/or potential treatment response using an EMG assessment alone typically requires a skilled practitioner and involves a subjective interpretation of the EMG data. The present combination of EMG data with an optical spectroscopy capability, such as Raman spectroscopy, provides an objective assessment of molecular composition of a target muscle region, and is therefore preferably suitable for use outside of clinical neurophysiology clinics for diagnostic, triage and disease monitoring purposes.

Optical spectroscopy data is precise with the potential to obtain highly sensitive and/or specific information, and can therefore preferably be used to improve, among other things, diagnosis and monitoring of disease progression.

In some embodiments, the muscle probe preferably further comprises a cannula, the cannula extending along the needle interior, the core electromyography electrode and/or the one or more optical fibres housed within the cannula. In preferable embodiments, the core electromyography electrode is formed from at least a part of the cannula. In some such embodiments, the cannula and the core electromyography electrode are the same. The cannula in preferable embodiments is electrically insulated from the outer wall of the needle, which may itself form an electrode of the needle. In such embodiments the cannula (which may itself be the core electromyography electrode) preferably comprises an electrically insulating coating.

The cannula may preferably be arranged to move along the needle interior. As such the cannula may act as an enclosed environment, separate to the needle outer wall, and may be permitted to move along the needle interior to engage a target muscle tissue, or target muscle area. Alternative embodiments will be appreciated wherein any component housed within the cannula, for example the active electrode and/or the one or more optical fibres, may move within the cannula and independently thereof, for engaging a target muscle tissue, or target muscle area. In some embodiments, it may be preferable for the core electromyography electrode to form a coating disposed on at least one said optical fibre. Coating one or more or more said optical fibres using the active electrode preferably further protects the coated optical fibres, and may improve ease of manufacture of the muscle probe.

The outer wall of the needle and/or the cannula may act as a protective layer for the active electrode and/or the one or more optical fibres contained therein. Said movement of the active electrode and the one or more optical fibres relative to the needle outer wall or cannula preferably enables the active electrode and/or the one or more optical fibres to remain protected within the needle interior, during movement of the needle toward a desired muscle tissue region. Said movement may then permit the active electrode and/or the one or more optical fibres to engage the muscle tissue for electromyography assessment or spectroscopic assessment.

In some embodiments, the one or more optical fibres preferably comprises: at least one delivery fibre arranged to direct the incident light from the light source toward the target area of the muscle; and at least one collection fibre arranged to receive the scattered light from the target area. In preferable embodiments, the one or more optical fibres comprise more collection fibres than delivery fibres. Such embodiments maximise collection area for receiving the scattered light from the target area, thereby optimising spectroscopic data collection. In some preferable embodiments, the one or more optical fibres comprise a single delivery fibre and at least three collection fibres. Such an arrangement may maximise collection capability while optimising form factor for use within a needle. Embodiments will be appreciated wherein any suitable number of collection and delivery fibres are used.

The one or more optical fibres may comprise in-line filters in some embodiments, for example to reduce the effect of compounding light (for example elastic or Rayleigh scattered light) or indeed the scattered light and any associated fluorescence, on the at least one delivery fibre, and additionally to reduce the effect of any such compounding light on the at least one collection fibre. In some embodiments, the at least one delivery fibre and/or the at least one collection fibre preferably comprises one of: an in-line short-pass filter; an in-line band-pass filter; an in-line long-pass filter; a notch filter. It will be appreciated that the terms “long-pass”, “short-pass”, “band-pass” and “notch” in the described filters, refer to the effects of the filters on light of particular wavelengths, rather than any relationship to a wave frequency.

In some particular embodiments, the at least one delivery fibre preferably comprises an in-line band-pass filter or an in-line short-pass filter. The filter on the at least one delivery fibre is preferably designed to only permit transmission of light at a particular wavelength (for example that of a light source, which may in some embodiments be a laser). The specific filter used may therefore be selected in accordance with the light source. In the case of a laser, a filter may be selected to substantially limit transmission of light to wavelengths of 785 nm or 830 nm. Embodiments will be appreciated wherein filters are used for any suitable wavelength of a light source. Such a filter preferably reduces the intensity of light that has been scattered inelastically within the fibre from reaching a target muscle region from which a spectroscopic measurement is to be made. Such a filter could be an in-line filter situated proximate the terminal of the at least one delivery fibre, or a filter coated directly onto or proximate said terminal end of the at least one delivery fibre. Filters coated directly onto said fibre may be easier to manufacture at scale. In embodiments wherein the scattered light comprises Raman scattered light, a band-pass or short-pass filter may be used to only observe Raman scattered light to longer wavelengths (Stokes shifted), but embodiments will be appreciated which are intended to observe Raman scattered light to both longer and/or shorter wavelengths (anti-Stokes shifted), wherein in such embodiments it may be preferable to include both a band-pass (or short-pass) filter and a notch-filter on the at least one delivery fibre.

In some particular embodiments, the at least one collection fibre preferably comprises an in-line long-pass filter or a notch filter. The filter on the at least one collection fibre is preferably designed to reject incoming light with a wavelength equal to that of a particular wavelength (of the light source), and may therefore be chosen according to the light source (for example that of a light source, which may in some embodiments be a laser). The specific filter used may therefore be selected in accordance with the light source. In the case of a laser, a filter may be selected to substantially limit transmission of light to wavelengths of 785 nm or 830 nm. This reduces the intensity of light that has been scattered elastically at the sample which is returned along the at least one collection fibre, where it would act as a source of Raman-and fluorescence-related scattering from the fibre itself. Such scattering would confound any intended spectroscopic measurement of light inelastically scattered from the target muscle region. Such a filter could be an in-line filter situated proximate the terminal end of the at least one collection fibre, or a filter coated directly onto, or proximate, said terminal of the at least one collection fibre. Filters coated directly onto said fibre may be easier to manufacture at scale.

In some embodiments, the needle outer wall preferably forms a conductive tube, the one or more optical fibres each comprising a light transmitting and/or light receiving end positioned proximate a terminal end of the conductive tube. Embodiments in which the needle is a monopolar electromyography needle, the needle outer wall may be comparatively non-electrically conductive. It will be understood that the optical fibres are included in the needle outer wall and/or the cannula such that the needle outer wall or the cannula encases at least the portion of the optical fibres intended to be inserted into a muscle, while permitting the required light emission and collection for optical spectroscopic assessment of the target muscle region. The needle outer wall and/or cannula therefore comprises an open terminal end such that incident light to be emitted by the one or more optical fibres is not occluded. The needle outer wall and/or cannula preferably provides protection for the optical fibres against damage during use, and without the needle outer wall and/or cannula such spectral data would therefore be difficult to obtain without significant hindrance and likelihood of complications. The needle outer wall may comprise any suitable conductive material, and in some embodiments, the outer wall of the needle preferably comprises steel. In embodiments comprising a monopolar electromyography needle, the outer wall of the needle may comprise any suitable polymer, such as polytetrafluoroethylene (PTFE). The core electrode may comprise any suitable material for performing the function of an active electrode of an electrography device, and in some embodiments, the core electrode preferably comprises any of: silver; platinum; steel; stainless steel; nickel; chromium; iridium; titanium; or any alloy thereof (for example nitinol or nichrome silver). In some embodiments, the muscle is preferably striated muscle or non-striated muscle (smooth muscle). In most preferable embodiments, the muscle is striated muscle.

An end of the one or more optical fibres opposing the light transmitting and/or light receiving end is preferably arranged for communication with an optical spectrometer. In some embodiments, the one or more optical fibres preferably comprise a silica core having a diameter selected from the range: 50 μm to 200 μm. Some preferable embodiments comprise a diameter of approximately 100 μm, for example 105 μm. In some embodiments, the one or more optical fibres preferably comprise a numerical aperture selected from the range: 0.2 to 0.3. Most preferable embodiments comprise a numerical aperture of approximately 0.2, for example 0.22. Such characteristics preferably combine an optimally large core diameter with an optimally large numerical aperture as possible for the at least one collection fibre, while considering form factor of the needle outer wall and/or a cannula, along with the physical constraints of any measurement devices.

In accordance with a second aspect of the present disclosure, there is provided a system for obtaining electromyography data and optical spectroscopy data from muscle, the system comprising: a muscle probe arranged to be inserted into a muscle, the muscle probe comprising a needle and one or more optical fibres; a light source arranged to provide incident light for transmission by the one or more optical fibres toward a target area of the muscle; an optical spectrometer arranged to receive scattered light from the one or more optical fibres; and an electromyography device arranged to receive an electrical signal from the needle; wherein the needle comprises an outer wall comprising a needle interior and a core electrode positioned within the needle interior, and wherein the one or more optical fibres are located within the needle interior.

In some embodiments, the muscle probe may be a muscle probe in accordance with the first aspect, and may therefore include any features described herein as being suitable for a muscle probe of the first aspect.

The one or more optical fibres may preferably comprise: at least one delivery fibre arranged to direct the incident light from the light source toward the target area of the muscle; and at least one collection fibre arranged to receive the scattered light from the target area. In some embodiments, each of the at least one delivery fibre and the at least one collection fibre each comprises one of: an in-line band-pass filter; an in-line short-pass filter; an in-line long-pass filter; a notch filter In some particular embodiments, the at least one delivery fibre preferably comprises an in-line band-pass filter or an in-line short-pass filter as described herein, and the at least one collection fibre comprises an in-line long-pass filter or a notch filter as described herein.

In some embodiments, the electromyography device is preferably configured to: determine, using the electrical signal, electromyography data (for example, recording of motor unit action potentials and other relevant waveforms); and the optical spectrometer is configured to: determine, using the received scattered light, optical spectra characteristic of the target area. In some embodiments, the system preferably further comprises a memory arranged to store the optical spectra and the electromyography data.

In some embodiments, the system preferably further comprises a processor, the processor arranged to perform one or more of: process the electromyography data and determine, using the electromyography data, the target area; and/or process the optical spectra, and optionally the electromyography data, and determine using the optical spectra and optionally the electromyography data, a data fingerprint of the target area. It will be appreciated that data fingerprint may comprise raw optical spectroscopy (and optionally EMG) data, or data following any suitable processing, such as to represent an average or distribution obtained from a series of data samples. The method may therefore provide an EMG-guided biochemical assessment of the target area, preferably reducing the time taken to achieve such an assessment, while combining the broad locating strength of EMG with the specificity, objectiveness, comparative quantitativeness, and non-invasiveness of spectroscopy. The data fingerprint may comprise data determined using optical spectroscopy or a combination of electromyography and optical spectroscopy. Such a fingerprint may comprise raw, pre-processed or processed optical spectra collected from the target area, and/or may comprise data representative of said spectra. In addition, the data fingerprint may comprise, or may be determined using, meta data, which may include any suitable meta data, but which may include for example one or more of: patient age, gender, symptoms, clinical examination findings. Other suitable meta data will be appreciated by the skilled addressee.

In some embodiments, the processor is preferably further arranged to: compare the data fingerprint of the target area with one or more stored data fingerprints; and determine, using said comparison, one or more of: an index of disease state; a prediction of disease state; a predicted disease prognosis; a predicted response to a treatment. Multivariate statistics may be used for such comparisons in example embodiments, as will be appreciated by the skilled addressee. In some embodiments, the processor preferably comprises a machine learning module trained using a plurality of stored fingerprints, the machine learning module arranged to process the data fingerprint and output one or more of: an index of disease state; a prediction of disease state; a predicted disease prognosis; a predicted response to a treatment.

In some embodiments, the light source is preferably a laser. In some embodiments, the incident light preferably comprises a wavelength selected from the near infra-red spectrum. In some particular embodiments, said wavelength may be selected from the range: 785 nm to 830 nm. These wavelengths preferably cause less fluorescence than visible wavelengths and so reduce any fluorescence spectrum that may be superimposed on to the intended optical spectrum, which could make the particular optical spectroscopy data (for example Raman data) difficult to measure. Such wavelengths are also preferably more biocompatible, since using short UV wavelengths could be mutagenic and cause tissue damage. Using longer infra-red wavelengths may create less spectroscopic (for example Raman) signal, and typically requires more expensive and less efficient detectors, than for example silicon-based detectors which may be used with the intended embodiments.

In accordance with a third aspect of the present disclosure, there is provided a computer-implemented method of: receiving, by the computer, an electrical signal from an electromyography needle, the electrical signal indicative of electrical activity in a muscle; determining, by the computer, based on the electrical signal, a target muscle location; outputting, by the computer the target muscle location for directing an optical spectroscopy probe to the target muscle location; and receiving, by the computer, optical spectroscopy data from the optical spectroscopy probe, the optical spectroscopy data characterising the target muscle location.

In some muscle pathologies, an electrical signal indicative of electrical activity of healthy muscle may be observed at the target muscle location. It will therefore be appreciated that in some embodiments, the electrical signal may be indicative of electrical activity of either healthy muscle or diseased muscle.

In accordance with a fourth aspect of the present disclosure, there is provided a computer-implemented method of: receiving, by the computer, optical spectroscopy data from the optical spectroscopy probe, the optical spectroscopy data characterising a muscle; determining, by the computer, based on the optical spectroscopy data, a target muscle location; outputting, by the computer the target muscle location for directing an electromyography needle to the target muscle location; and receiving, by the computer, an electrical signal from an electromyography needle, the electrical signal indicative of electrical activity in the muscle at the target muscle location.

In some embodiments, the method or the third or fourth aspect preferably further comprises: determining, based on the optical spectroscopy data, and optionally further based on the electrical signal, one or more of: an index of disease state; a prediction of disease state; a predicted disease prognosis; a predicted and/or measured response to a treatment. It will be appreciated that the electric signal may be processed by, for example, an electromyography device to provide electromyography data (for example a recording of motor unit action potentials and other relevant waveforms), ahead of use said determining step. The available processes of performing said determination can include any suitable process, such as by comparing the optical spectroscopy data, and optionally the electric signal, to stored optical spectroscopy data, and optionally stored electrical signal data. Other processes may include utilising a machine learning module trained on stored optical spectroscopy data, and optionally stored electrical signal data, to perform said determining step. Such a determination may, in some embodiments, involve the generation, by the processor, of a digital fingerprint using the optical spectroscopy data, and optionally the electrical signal (or data obtained therefrom such as optical spectra or electromyography data). Such a fingerprint may represent a digital biomarker characterising one or more of: one or more neuromuscular diseases; a prognosis of the muscle and/or a disease associated therewith; an index of response of the muscle (and/or a disease associated therewith) to a treatment.

In some embodiments, said determination is preferably performed by processing the optical spectroscopy data, and optionally the electrical signal, using a machine learning module trained using stored optical spectroscopy data, and optionally stored electrical signals.

The method may in some embodiments be performed using a muscle probe in accordance with the first aspect, or a system in accordance with the second aspect.

In accordance with a fifth aspect of the present disclosure, there is provided a method of determining a muscle pathology (such as, for example, acute myopathy, chronic myopathy, inflammatory myopathy; dystrophic myopathy; mitochondrial myopathy; a neurogenic muscle pathology, or any suitable pathology as discussed herein) at a target muscle location of a test subject, the method comprising: at a computer system comprising at least one processor and a memory storing at least one program for execution by the at least one processor, the at least one program comprising instructions for: obtaining a dataset, in electronic form, wherein the dataset comprises a test optical spectroscopy data sample obtained from a target muscle location of a test subject; and applying the dataset to a machine learning classifier trained using stored optical spectroscopy data, thereby determining the muscle pathology in the test subject.

In some embodiments, the optical spectroscopy data sample is preferably determined using one or more of: Raman scattering; fluorescence scattering; Brillouin scattering at the target muscle location of the test subject

In some embodiments, the program preferably further comprises instructions for: isolating, from the test optical spectroscopy data sample, a spectral region comprising spectral data characterising at least one protein secondary structure. In some embodiments, the spectral region is preferably obtained from the amide I band. Any suitable spectral region will be appreciated, the region preferably associated with muscle protein, and more preferably associated with protein secondary structures. Limiting the optical spectroscopy data to that associated with muscle, or more specifically to protein secondary structures, preferably provides more relevant spectral data to the classifier and thereby preferably results in an improved false discovery rate.

The spectral data preferably characterises at least an amount or a proportion (which may be relative or quantitative) of at least one protein secondary structure at the target muscle location, and preferably of alpha helix at the target muscle location, and of beta sheet at the target muscle location. The spectral data may, in some embodiments characterise a said amount or proportion of non-regular secondary structure at the target muscle location.

The target muscle location, in some embodiments, is preferably determined using an electrical signal from an electromyography needle, the electrical signal indicative of an electrical activity in a muscle. The electrical signal and/or the electrical activity is preferably one indicative of muscular pathology. In some muscle pathologies, the electrical signal may be indicative of electrical activity observed in healthy muscle. Embodiments will therefore be appreciated wherein the electrical signal and/or the electrical activity is preferably one indicative of healthy muscle. In some embodiments, the predetermined threshold is preferably associated with an electrical activity of a healthy muscle. In some embodiments, the dataset further comprises test electrical signal data from an electromyography needle, the test electrical signal data indicative of electrical activity at the target muscle location of the test subject; and further wherein the machine learning classifier is further trained using stored electrical signal data. The electrical signal data preferably comprises data indicative of motor unit action potentials such as motor unit action potential morphology or configuration, motor unit action potential recruitment, and/or data indicative of spontaneous muscle cell activity. Embodiments will be appreciated wherein the electrical signal data preferably comprises data indicative of compound muscle action potential (CMAP) amplitudes at the target muscle location.

In preferable embodiments, the machine learning classifier comprises, or is generated using, any suitable embedding model or dimensional reduction technique. In some embodiments, the machine learning classifier comprises, or is generated using matrix factorisation. Matrix factorisation has been identified as a powerful machine learning technique for identifying muscle pathology, and preferably improves interpretation of results at the individual sample level, rather than requiring group analysis, providing a desirable addition to the diagnostic pathway. The term “sample” as used herein will be understood to mean sample data obtained from a subject, and may include a group of data obtained from the subject, or a single data entity for said subject, for example a single spectrum. In particular, matrix factorisation provides a more interpretable output than traditionally used methods. Suitable matrix factorisation methods may include, for example, non-negative matrix factorisation or bounded simplex-structured matrix factorization (BSSMF). In some particular embodiments, relevant protein secondary structure information is determined through matrix factorisation of a dataset comprising optical spectroscopy data obtained from a plurality of training subjects (such as comprising healthy and diseased subjects). In particular, matrix factorisation is used to identify dominant spectral patterns within a dataset, each training data sample preferably being allocated a corresponding weight for each said spectral pattern, the corresponding weights associated with the relative importance of said spectral pattern to the training data sample. Preferably each spectral pattern is associated with a corresponding protein secondary structure. In some embodiments therefore, said corresponding weights are calculated by integrating the area under the relevant spectral data region, such as that relating to a-helix (1650-1658 cm⁻¹), B-sheet (1664-1673 cm⁻¹) and nonregular (1630-1640, 1674-1689 and 1700-1710 cm⁻¹). Said weights may be applied to a model, such as a linear discriminant model, for classification of the test dataset.

In some embodiments, the machine learning classifier comprises, or is generated using hierarchical modelling. The use of hierarchical modelling, for example to provide an expert systems approach, has been identified to improve false discovery rate at the single sample level, while also maintaining power to provide successive classifications which may provide more useful tool in a clinical setting. This may, for example, include the use of a hierarchical classification model constructed based on multiple successive classification problems relevant to a clinical setting in determining the presence of a muscle pathology such as myopathy, a sub-class of myopathy (such as acute or chronic myopathy), or a neurogenic muscle pathology. For example, the model may first distinguish a “diseased” sample from a “healthy” sample, and may subsequently classify the “diseased” sample in accordance with a disease type, followed by a more specific disease sub-type. For example, a model may be configured to first determine “diseased” samples from “healthy” samples, and to subsequently determine a “myopathy” sample a “neurogenic pathology” sample, and to subsequently determine “acute myopathy” sample from a “chronic myopathy” samples. It will be understood that any suitable classification may be performed in accordance with the training data used to train the classifier. As an example, successive two-class problems may be presented, such as “healthy” vs “disease” (wherein “disease” encompasses a sample data group from test subjects exhibiting one of acute myopathy, chronic myopathy and a neurogenic condition); “myopathy” (encompassing a sample data group from test subjects exhibiting either acute myopathy or chronic myopathy) vs “neurogenic”; and “acute myopathy” vs “chronic myopathy”. These hierarchical classifications may be trained using any suitable means, for example using a partial least squares discriminant analysis (PLS-DA) model constructed using the training dataset for each of the relevant two-class problems. In some embodiments, the training may comprise a feature selection, for example using the variable importance in projection approach, followed by selection of a reduced number of variables selected for inclusion in the models. The hierarchical modelling approach therefore provides improved classification flexibility while maintaining improved performance in assessing individual samples. The hierarchical modelling approach additionally more closely approximates decisions required in clinical practice and therefore improves applicability for the clinical setting.

In some embodiments, the machine learning classifier comprises, or is generated using multi-block modelling or data fusion modelling, trained by any suitable means as described herein, such as using a PLS-DA model. In preferable such embodiments, the machine learning classifier is further trained using electrical signal data indicative of an electrical activity within the target muscle region (for example motor unit action potentials and/or data indicative of spontaneous muscle fibre activity), and wherein the test dataset includes said electrical signal data obtained from the target muscle region. In particular, when combining optical spectroscopy data and said electrical signal data, the predictive power of the multi-block modelling or data fusion modelling approach is improved, thereby maintaining optimal false discovery rate without having to increase the amount of training data. Embodiments will be appreciated wherein the machine learning classifier is generated using any combination of: matrix factorisation; principal component analysis; hierarchical modelling; multi-block modelling; data fusion modelling. In preferable embodiments, the classification may include any suitable disease or disease subtype, for example muscle myopathy or a neurogenic pathology, wherein the muscle myopathy may be any one selected from: acute muscle myopathy; chronic muscle myopathy; inflammatory myopathy; immune myopathy; dystrophic myopathy; mitochondrial myopathy; inherited myopathy; congenital myopathy; metabolic myopathy; toxic myopathy; endocrine myopathy; infectious myopathy; critical illness myopathy; muscular dystrophy; neurogenic pathology.

In accordance with a sixth aspect of the present disclosure, there is provided a digital biomarker determined using either optical spectroscopy data obtained from a muscle, or a combination of optical spectroscopy data and electromyography data obtained from a muscle, the digital biomarker characterising one or more of: one or more neuromuscular diseases; a prognosis of the muscle and/or a disease associated therewith; an index of response of the muscle (and/or a disease associated therewith) to a treatment. The digital biomarker is preferably determined using a muscle probe in accordance with the first aspect, a system in accordance with the second aspect, or a method in accordance with the third aspect.

The optical spectroscopy data may in preferable embodiments be any data obtained by analysing scattered light received from a target muscle tissue region. Said scattered light is preferably inelastic scattered light. The optical spectroscopy is preferably one or more of: Raman spectroscopy; fluorescence spectroscopy; Brillouin spectroscopy.

It has been observed that neuromuscular disorders and/or myopathy can be identified in accordance with an altering of protein secondary structures within an affected muscle. Therefore, in preferable embodiments, the optical spectroscopy data preferably characterises amounts or proportions, such as relative or quantitative amounts or proportions, of any suitable one or more secondary structures at the target muscle region, which may include, for example, alpha helix, beta sheet, and/or non-regular secondary structures. In preferable embodiments, the optical spectroscopy data preferably characterises a proportion of alpha helix and a proportion of beta sheet of said muscle.

Other embodiments will be appreciated wherein the optical spectroscopy data preferably characterises a proportion of non-regular secondary structure to alpha helix and/or beta sheet. In many cases muscle pathologies can be identified in accordance with a proportional increase or reduction of a particular protein secondary structure relative to other protein secondary structures at a target muscle region. In the case of neuromuscular disorders and/or myopathy, a reduction in alpha helix relative to beta sheet within the muscle is often observed. Preferably therefore, the proportion of alpha helix relative to the proportion of beta sheet is below a predetermined threshold. In some embodiments, said predetermined threshold is a proportion of alpha helix relative to a proportion of beta sheet associated with a healthy muscle. In some embodiments, said predetermined threshold is a ratio of alpha helix relative to beta sheet of less than 1. The proportional reduction of alpha helix relative to beta sheet in a target muscle region, when compared to a healthy muscle, has been identified as indicative of neuromuscular disorders and/or myopathy. Preferably said predetermined threshold is determined by a machine learning module trained on stored optical spectroscopy data obtained from muscle, or a combination of stored optical spectroscopy data and stored electromyography data obtained from muscle.

The digital biomarker may be further determined using, or comprise, meta data, which may include any suitable meta data, but which may include for example one or more of: patient age, gender, symptoms, clinical examination findings. Other suitable meta data will be appreciated by the skilled addressee.

In accordance with a seventh aspect of the present invention, there is provided a non-transitory computer readable storage medium storing a digital biomarker in accordance with the sixth aspect.

It will be appreciated that any features described herein as being suitable for incorporation into one or more aspects or embodiments of the present disclosure are intended to be generalizable across any and all aspects and embodiments of the disclosure.

### DETAILED DESCRIPTION

Embodiments of the present disclosure will now be described by way of example only and with reference to the accompanying drawings, in which:

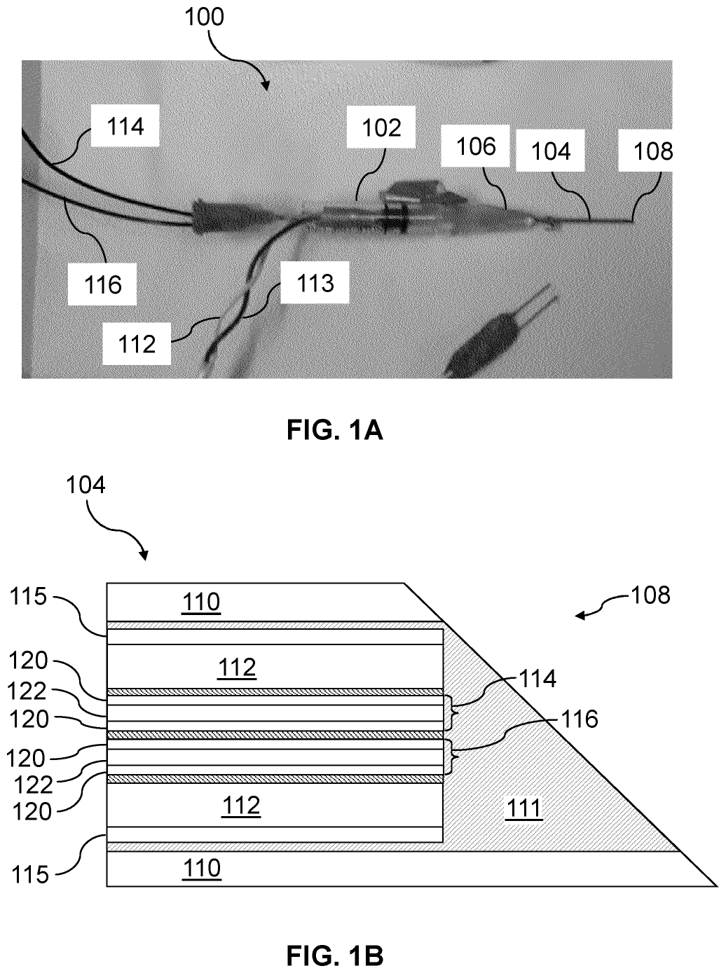

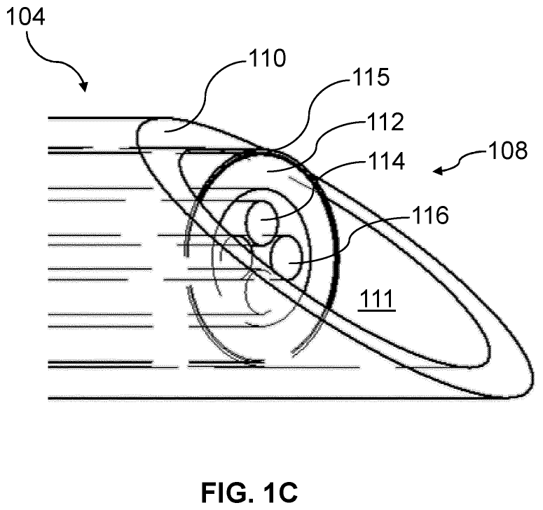

FIG. 1A shows a perspective view of an example muscle probe in accordance with the first aspect of the present disclosure;

FIG. 1B shows an expanded cut-away view of the terminal end of the needle of the embodiment shown in FIG. 1A;

FIG. 1C shows a perspective cut-away view of the terminal end of the needle of the embodiment shown in FIG. 1A and FIG. 1B;

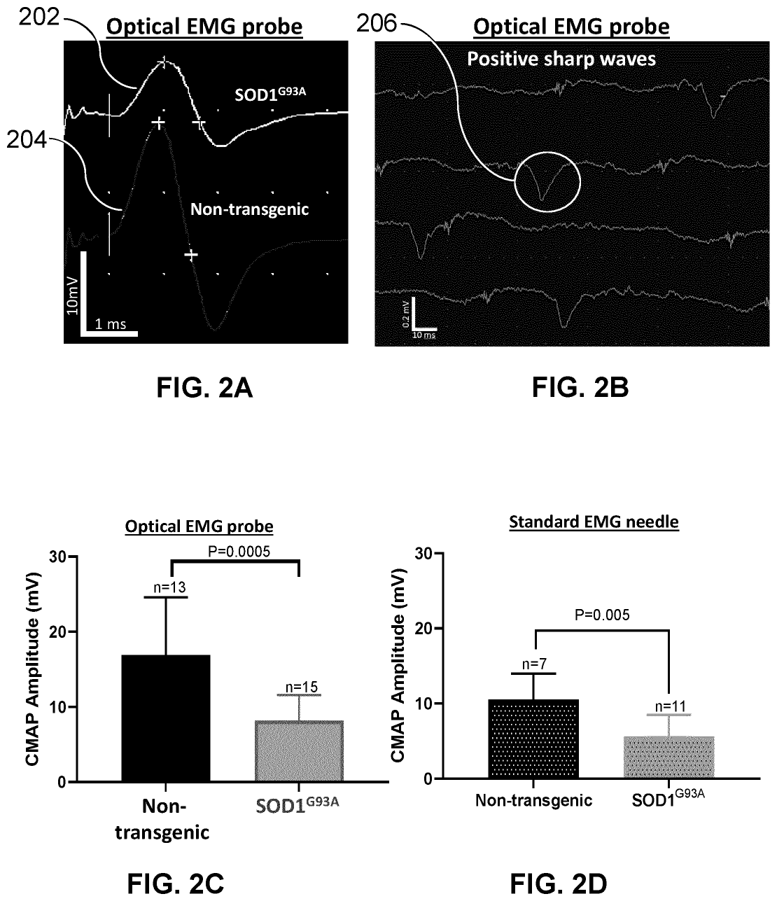

FIG. 2A to FIG. 2D shows results of an experiment demonstrating electrophysiological functionality of a probe in accordance with the first aspect, in a SOD1G93A mouse model at 90 days of age; in particular FIG. 2A shows compound muscle action potential (CMAP) waveforms from both SOD1G93A and non-transgenic (NTg) mice using the optical EMG probe; FIG. 2B shows spontaneous EMG activity (positive sharp waves; example circled) from the optical EMG probe; FIG. 2C shows a comparison of a CMAP amplitude in non-transgenic (NTg) and SOD1G93A mice using the optical EMG probe; and FIG. 2D shows a comparison of a CMAP amplitude in NTg and SOD1G93A mice using a standard EMG needle;

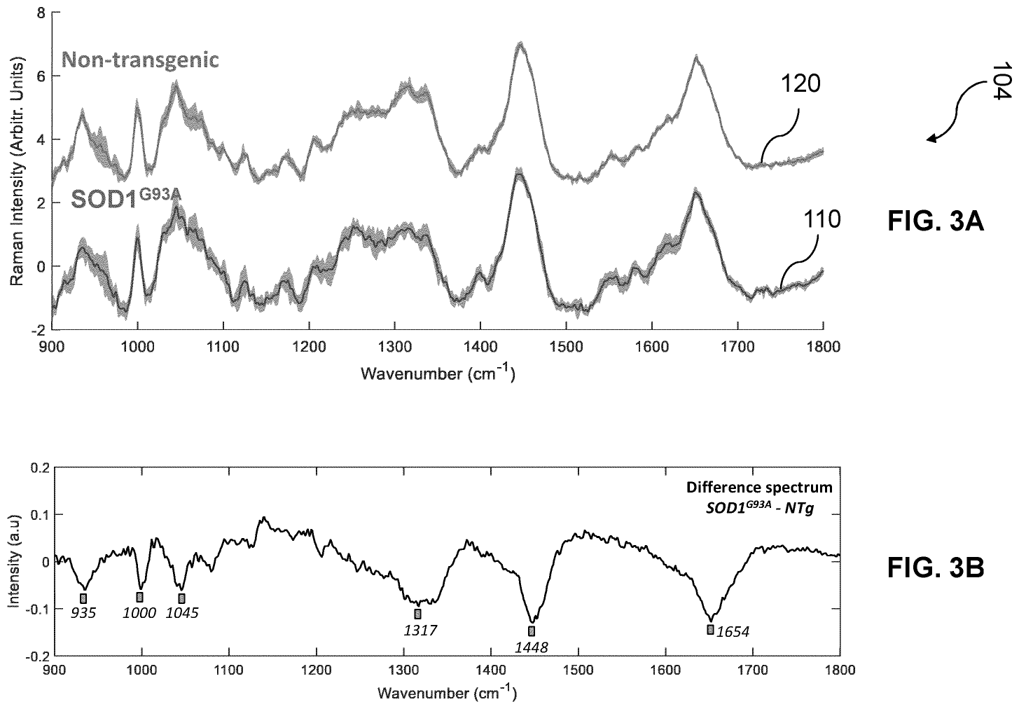

FIG. 3A and FIG. 3B shows example spectroscopy results (which in the specific example described is Raman spectroscopy) from an experiment utilising a muscle probe in accordance with the first aspect to obtain Raman spectroscopy data; in particular FIG. 3A shows mean Raman spectra (±standard deviation) for NTg (top) and SOD1G93A mice (bottom), both at 90-days; and FIG. 2B shows a difference of the mean spectrum (SOD1G93A minus NTg);

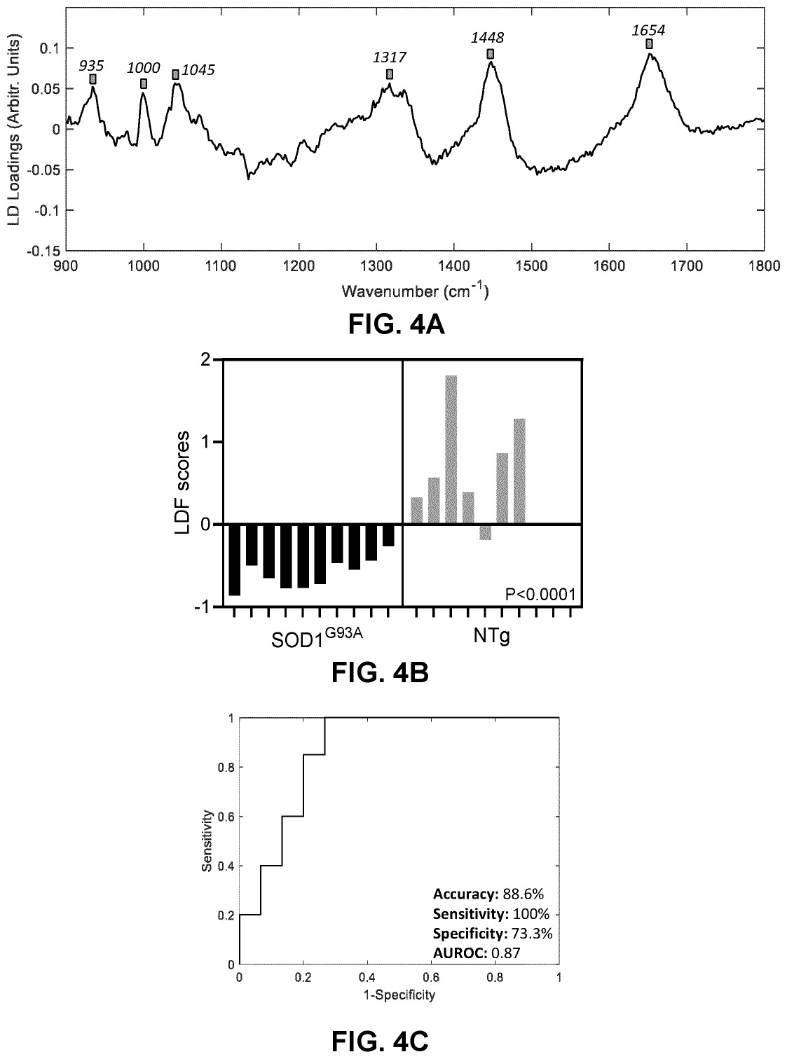

FIG. 4A to FIG. 4C shows results of a multivariate analysis of Raman spectroscopy data obtained using a muscle probe in accordance with the first aspect; in particular FIG. 4A shows the loadings plot of the linear discriminant. The wavenumbers of the more prominent peaks are labelled, these contribute the most to the SOD1G93A vs. NTg classification; FIG. 4B shows average LDF scores for each mouse were significantly different at a group level (nested t-test); and FIG. 4C shows a receiver operator characteristic curve and classification performance data for a PCA-LDA model;

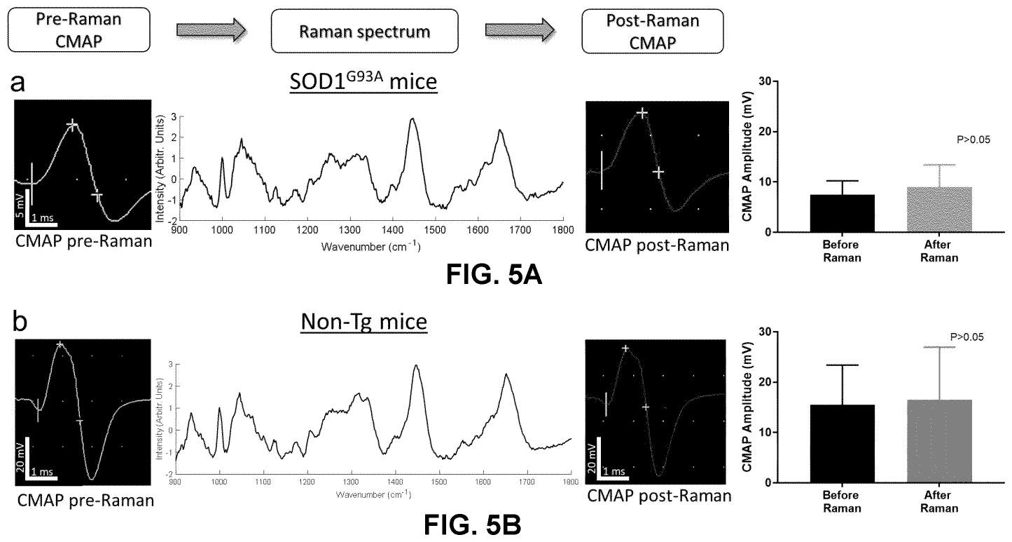

FIG. 5A and FIG. 5B shows that in vivo intra-muscular Raman spectroscopy does not alter CMAP amplitudes; in particular FIG. 5A shows example CMAPs and Raman spectra from SOD1G93A mice, recorded using the optical EMG probe—no significant difference was seen between the pre- and post-Raman CMAP amplitudes; and FIG. 5B shows example CMAPs and Raman spectra from NTg mice—no significant difference was seen between the pre- and post-Raman CMAP amplitudes;

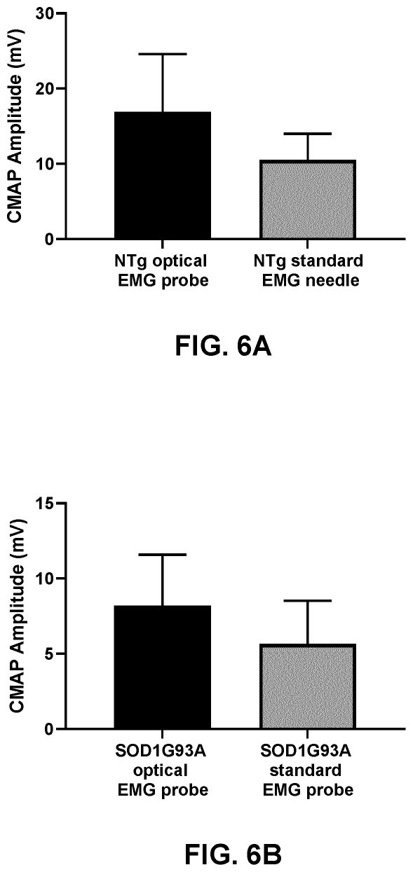

FIG. 6A and FIG. 6B shows a comparison between a muscle probe in accordance with the first aspect (optical EMG probe), and standard EMG needle CMAP amplitudes; in particular FIG. 6A shows CMAP amplitudes were slightly smaller with the standard EMG needle but this did not reach statistical significance (P=0.05); and FIG. 6B shows that CMAP amplitudes were not significantly different in SOD1G93A mice;

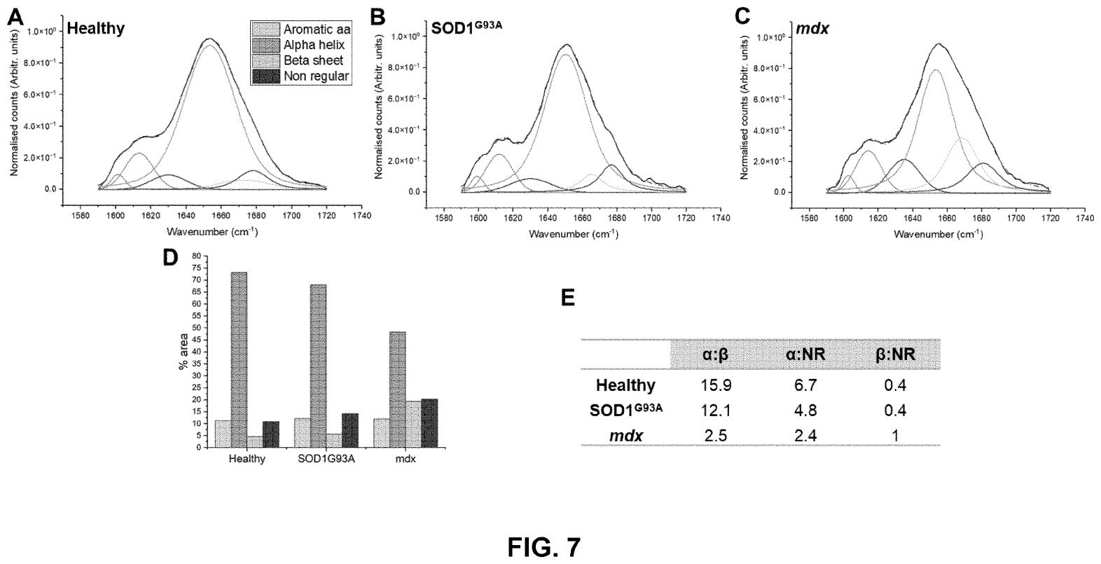

FIG. 7 shows preclinical peak fitting of healthy, myopathic and neurogenic muscle shows a reduction in alpha helix conformation in myopathy. In particular, FIG. 7A to 7C shows peak fitting using standard peaks within the amide 1 region for healthy, mdx and SOD1^{G93A} mice. FIG. 7D shows each of the peaks resolved as a percentage of the total area. A reduction of a-helix and concomitant increase in β-sheet can be seen in the model of myopathy (mdx). FIG. 7E shows ratios of different protein secondary structure conformations. The reduced α-helix/increased β-sheet is evident in the mdx model of myopathy, which also manifests an increase in the β-sheet/nonregular ratio;

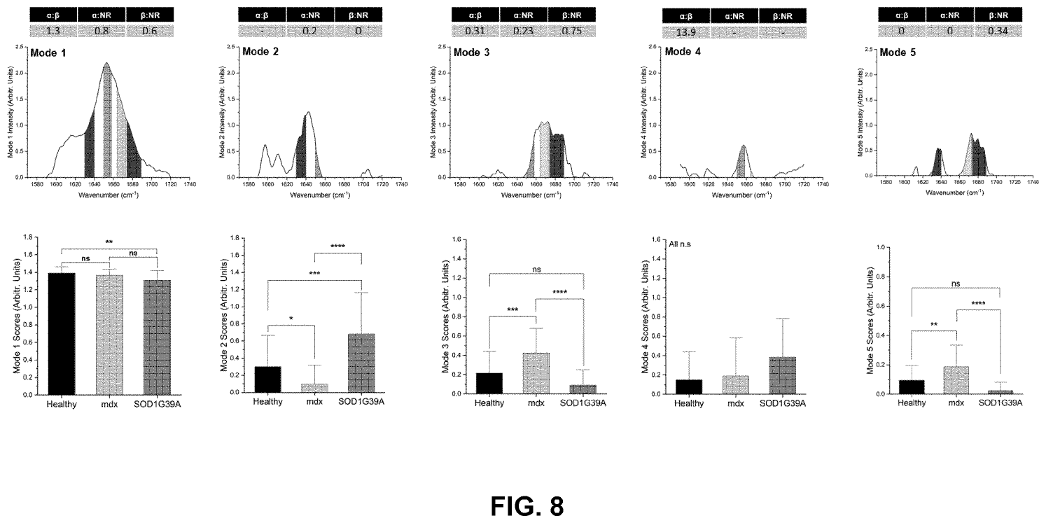

FIG. 8 shows non-negative matrix factorisation derived spectral patterns show conformational differences. The unique spectral patterns output from the non-negative matrix factorisation (left) manifest differences in protein structure (middle) and differences between healthy, mdx and SOD1^{G93A} mice (right). The different conformational fingerprints and their predominance in different mice matches those seen via peak fitting (*P<0.05, **P<0.01, ***P<0.001, ****P<0.0001);

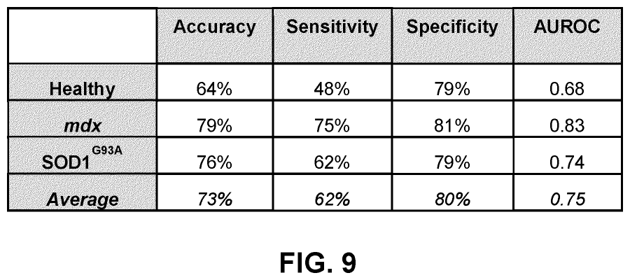

FIG. 9 provides a table showing three-group, cross validated classification performance for in vivo preclinical data using the non-negative matrix factorisation modes and a linear discriminant model;

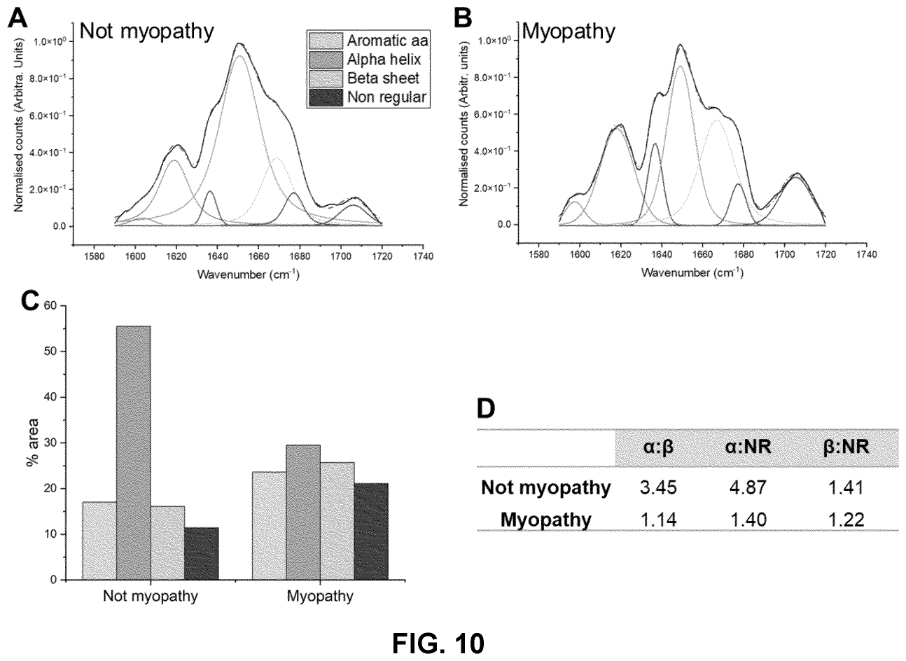

FIG. 10 shows peak fitting from Raman muscle spectra from human samples obtained from patients with and without myopathy. In particular, FIGS. 10A and B show peak fitting within the amide I region. FIG. 10C shows each of the peaks resolved as a percentage of the total area. A reduction of α-helix and increases in other conformations and aromatic amino acids can be seen in the myopathy group. FIG. 10D shows ratios of different protein conformations. The reduction in α-helix and increased β-sheet/nonregular structures is evident in myopathy;

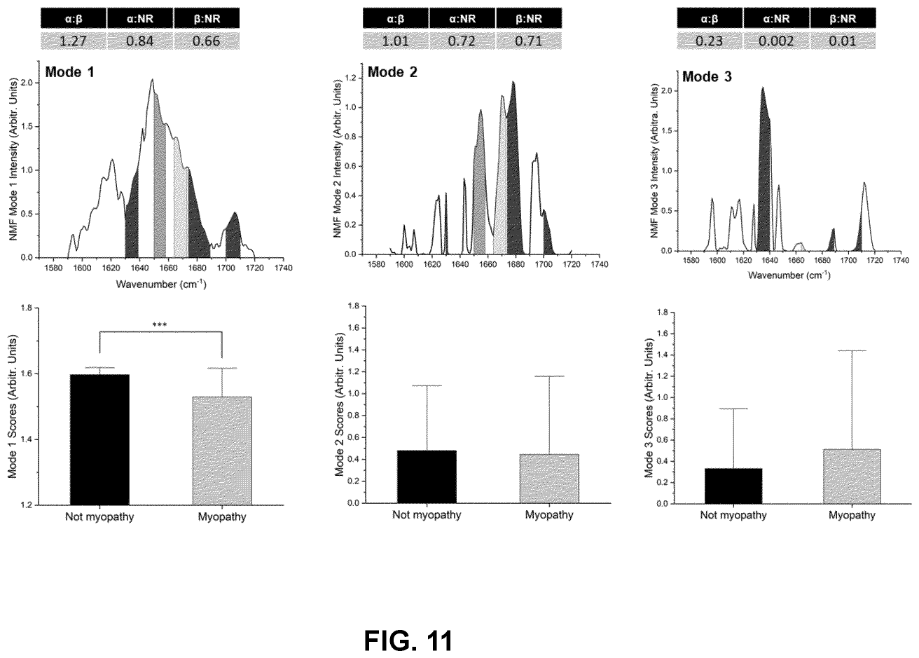

FIG. 11 shows non-negative matrix factorisation derived spectral patterns in human muscle. In particular, the unique spectral patterns output from the non-negative matrix factorisation (left) manifest differences in protein structure (middle). A significant difference was observed for mode 1 (*** P<0.001);

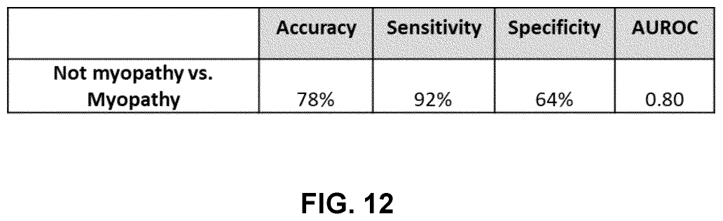

FIG. 12 provides a table showing two-group classification performance for human ex vivo samples using the non-negative matrix factorisation modes and a linear discriminant model;

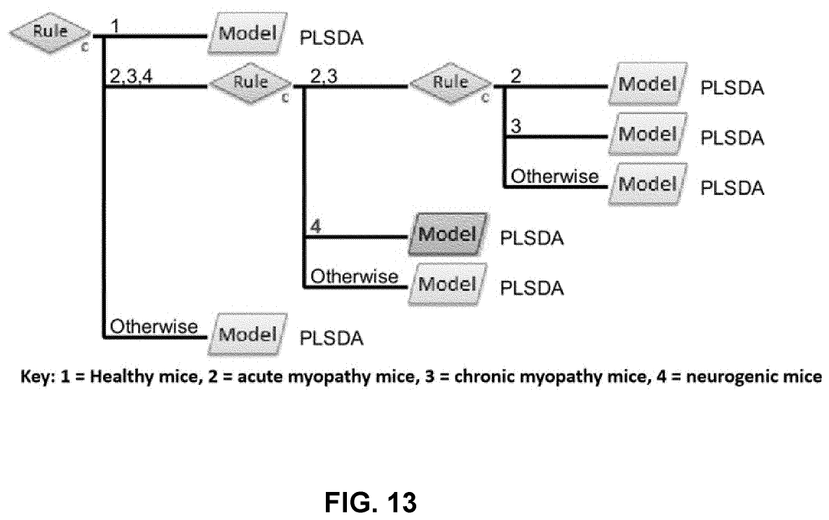

FIG. 13 shows a schematic view of a hierarchical model suitable for use in a method in accordance with the fifth aspect;

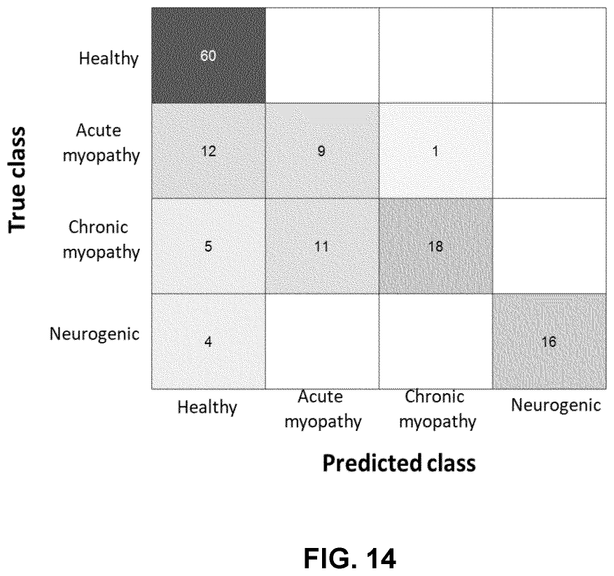

FIG. 14 shows a confusion matrix indicating classification performance for individual spectra derived from the hierarchical model approach shown schematically in FIG. 13, with data obtained from mice (healthy/non-transgenic mice=healthy; acute myopathy=30 day-old mdx; chronic myopathy=90 day-old mdx mice; neurogenic=90 day-old SOD1G93A mice);

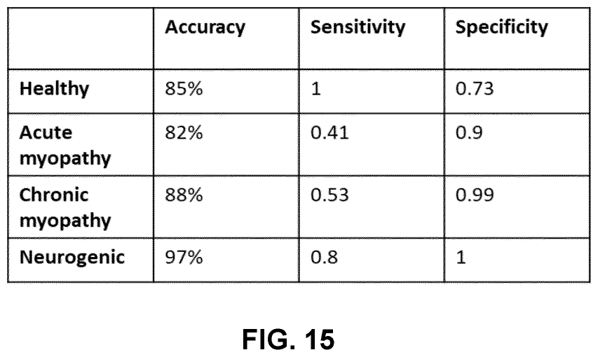

FIG. 15 shows a table indicating classification performance statistics for validation test data derived from the hierarchical model approach shown schematically in FIG. 13;

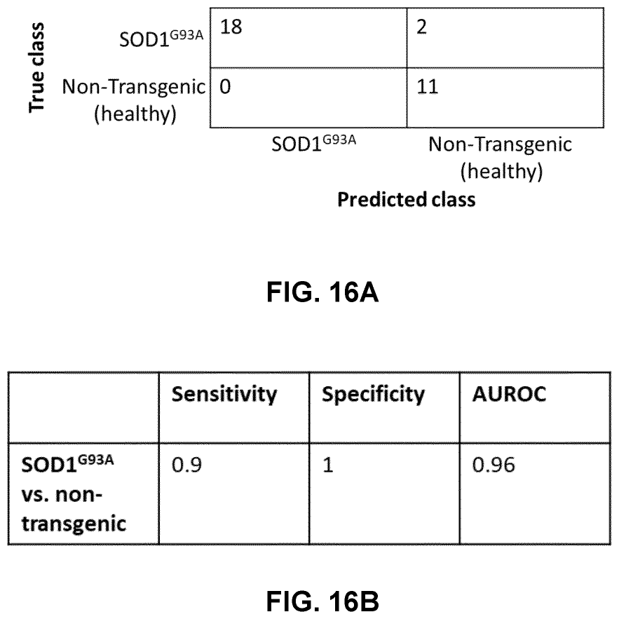

FIG. 16A shows a confusion matrix indicating classification performance for a multi-block model suitable for use in a method in accordance with the fifth aspect, the multi-block model making use of Raman spectroscopy data combined with CMAP data;

FIG. 16B shows a table indicating classification performance statistics for the multi-block model approach of FIG. 16A;

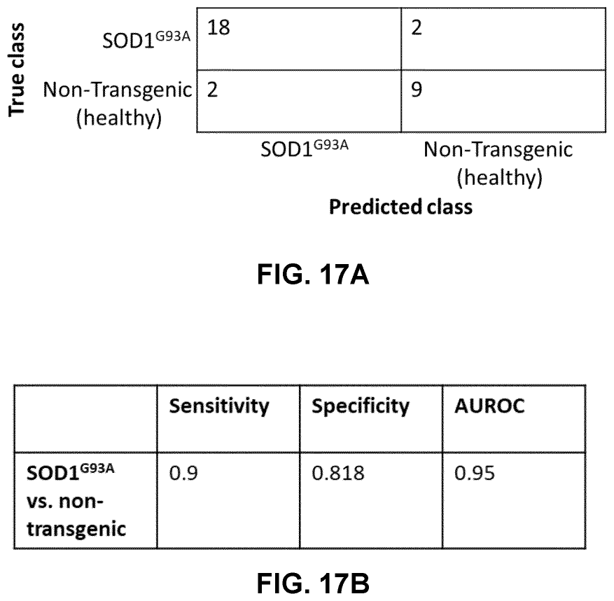

FIG. 17A shows a confusion matrix indicating classification performance for a model suitable for use in a method in accordance with the fifth aspect, the model making use of Raman spectroscopy data alone;

FIG. 17B shows a table indicating classification performance statistics for the model approach of FIG. 17A;

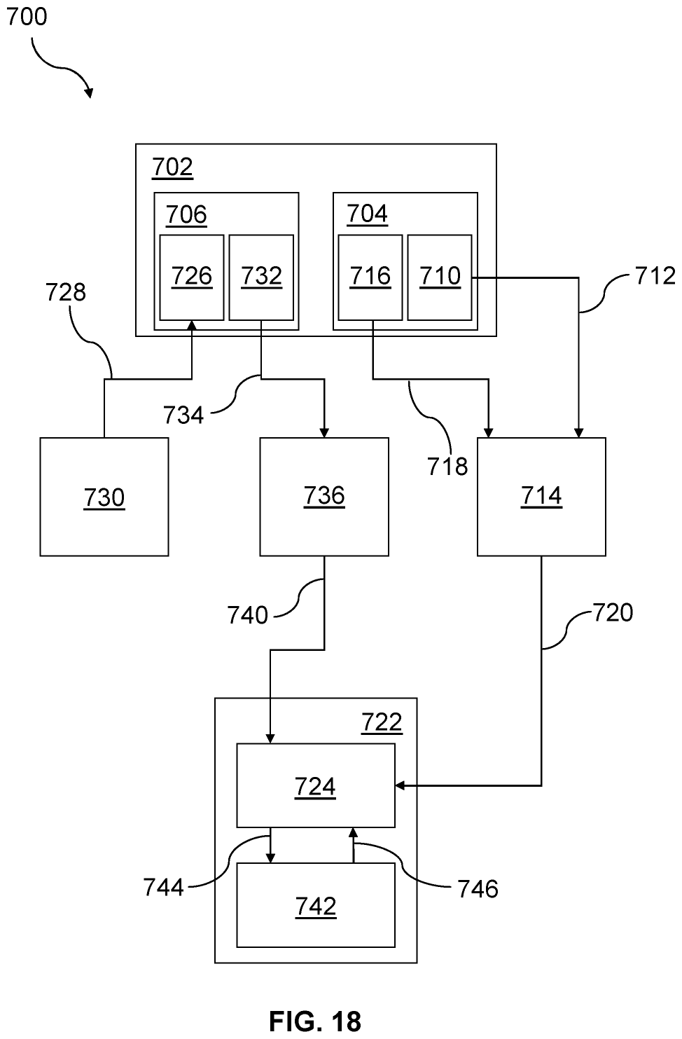

FIG. 18 shows a schematic view of an example embodiment of a system in accordance with the second aspect of the present disclosure; and

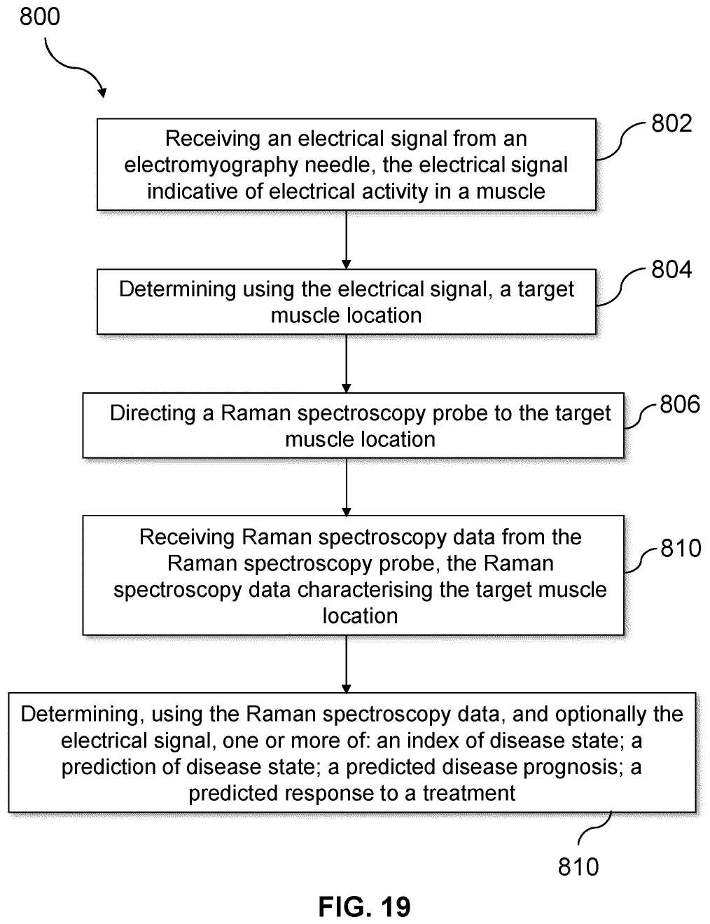

FIG. 19 shows a flow chart listing steps of an example embodiment of a method in accordance with the third aspect of the present disclosure.

With reference to FIG. 1A, a perspective view of an example muscle probe 100 in accordance with the first aspect of the present disclosure is shown. The probe 100 comprises a probe body 102 having a needle 104 affixed thereto by way of a standard luer connector 106. The needle 104 comprises an elongate steel outer wall 110 extending from the luer connector 106 and terminating at an open terminal end 108 distal to the probe body 102.

FIG. 1B and FIG. 1C each show a close-up cutaway view of a portion of the needle 104 proximate the terminal end 108 thereof. As can be seen in FIG. 1B, the needle comprises a tubular outer wall 110 defining a needle interior 111 therein. In the particular example shown, the outer wall 110 is formed from a standard 21G hypodermic needle with an outer diameter of 0.819 mm, an inner diameter of 0.514 mm and a terminal bevel angle of 12 degrees relative to a plane parallel to the longitudinal axis of the outer wall 110, to provide a needle point. In use, the needle point is used to insert the outer wall 110 into muscle tissue (not shown).

The needle interior 111 houses a silver electrode 112 extending therealong, the electrode 112 in the example embodiment shown forming a tube. The electrode 112 in the embodiment shown is coated by a polymer coating 115 acting to electrically insulate the electrode 112 from the needle outer wall 110. A small terminal region of the electrode 112 remains uncoated in order to obtain a required electromyography signal from a target muscle region in use.

The needle outer wall 110 comprises a conductive wire 113 extending therefrom. The conductive wire 113 and the electrode 112 each extend along the probe body 102, out of an end of the probe body 102 distal to the needle 104, to an electromyograph (not shown). In use, each of the electrode 112 and the conductive wire 113 (connected to the conductive needle outer wall 110) are arranged to transmit an electrical signal from the muscle tissue to the electromyograph. The electromyograph then processes the electrical signals to provide a recording of motor unit action potentials and other relevant waveforms as will be understood by the skilled addressee.

The interior of the tubular electrode 112 houses a plurality of optical fibres 114, 116, which in the embodiment shown comprise a light emitting optical fibre 114 and three light receiving optical fibres 116. Each optical fibre 114, 116 comprises a cladding layer 120 encasing a light propagating core 122. Each of the optical fibres 114, 116 in the embodiment shown comprise low-OH fibres, having a silica core of diameter 105 um and a numerical aperture (NA) of 0.22. For explanatory purposes the example 100 shown comprises a single light emitting optical fibre 114 and three light receiving optical fibres 116. Embodiments will be appreciated comprising any number of light emitting optical fibres and light receiving optical fibres. In order to maximise light collection area, embodiments preferably comprise more light receiving optical fibres than light emitting optical fibres. Embodiments will be appreciated wherein a single optical fibre is used for light emission and light receiving.

Each of the light emitting optical fibre 114 and the light receiving optical fibres 116 extends through the interior of the tubular electrode 112, and further through the length of the probe body 102, and out of an end of the probe body 102 distal to the needle 104. The light emitting optical fibre 114 extends to a light source, which in the embodiment described is a semiconductor laser (not shown). Positioned proximate to the terminal end of the light emitting optical fibre 114 shown (approximately 15 cm from said terminal end in the particular example shown), the light emitting optical fibre 114 comprises a bandpass filter (not shown). The light receiving optical fibres 116 extend to a Raman spectrometer (not shown). Between the probe body 102 and the Raman spectrometer, positioned proximate the terminal end of the light receiving optical fibres 116 shown (approximately 15 cm from said terminal end in the particular example shown), the light receiving optical fibres 116 comprise a long-pass filter (not shown). In use, the semiconductor laser emits a light beam, which in the embodiment shown comprises a wavelength of 830 nm. The light beam is propagated along the core 122 of the light emitting optical fibre 114 toward the muscle. Raman scattered light reflected from the muscle is received by an end of the light receiving optical fibres 116 proximate the terminal end 108 of the needle 104. The Raman scattered light propagates along the core 122 of the light receiving fibre 116 to be transmitted to the Raman spectrometer. The Raman spectrometer processes the received light to provide a Raman spectra characterising the molecular composition of the muscle area targeted by the light emitting optical fibre 114. The Raman spectra, and optionally the recording of motor unit action potentials and other relevant waveforms, may be used as a digital biological fingerprint of the assessed muscle region, which may be used to determine pathology, prognosis, disease progression, an expected response to treatment, among other suitable clinical outcome measures.

FIG. 2A to FIG. 6B are described hereinafter and show results of experimentation using a muscle probe in accordance with the first aspect, and as shown in FIG. 1A and FIG. 1B. Such a muscle probe is suitable for use in a system in accordance with the second aspect to perform a method in accordance with the third aspect, providing a digital biomarker in accordance with the fourth aspect. The following description outlines: 1. the methodology used in collecting the data which is represented in FIG. 2A to FIG. 6B; 2. the results obtained from the experimentation; and 3. the conclusions drawn from said results.

### Methodology

### Raman Spectroscopy

For Raman data collection, a probe substantially as described above in relation to FIG. 1A and FIG. 1B (referred to hereinafter as “the optical EMG probe”) was provided, and connected to the 830 nm semiconductor laser (Innovative Photonics Solutions) as described, which was used to provide a superior signal/noise ratio. In-line filters (Semrock, Inc) described in relation to FIG. 1A and FIG. 1B were used to reduce the effect of the Raman signal and associated fluorescence on the light emitting optical fibres. The laser power was 60 mW at the probe tip. The optical EMG probe was optically matched to a Raman spectrometer (Raman Explorer Spectrograph, Headwall Photonics, Inc. and iDus 420BR-DD CCD camera, Andor Technology, Ltd.). The Raman signal was recorded through a 40 second exposure consisting of 10×4 second epochs which were averaged. Spectra from polytetrafluoroethylene (PTFE) were acquired for wavenumber calibration of the spectrometer.

### Electromyography

For electrophysiological data collection the optical EMG probe was connected to a Dantec Keypoint Focus EMG system with standard filter settings (20 Hz-10 kHz). For comparison, compound muscle action potential (CMAP) recordings were also made using a probe comprising a standard commercially-available concentric EMG needle (Ambu Neuroline, 30G) (referred to hereinafter as “the concentric EMG needle”).

### In Vivo Testing

Mouse experiments were carried out in accordance with the Animals (Scientific Procedures) Act 1986, under a UK Home Office project licence (number 70/8587). The project was approved by the by the University of Sheffield Animal Welfare and Ethical Review Body (AWERB). Mice were housed in a standard facility (12-hour light/dark cycle, room temperature 21° C.) and cared for in accordance with the Home Office Code of Practice for Housing and Care of Animals Used in Scientific Procedures. The ARRIVE guidelines were followed in the conduct of this work.

Transgenic C57BL/6J-Tg (SOD1G93A) 1Gur/J mice were used (originally obtained from Jackson Laboratories). Hemizygous transgenic males were backcrossed to C57BL/6 females (Harlan UK, C57BL/6 J OlaHsd substrain) for over 20 generations. Hemizygous females were used for experiments, with non-transgenic (NTg) females used as controls. Transgenic SOD1G93A mice were identified through PCR amplification of genomic DNA extracted from ear clips. The mice are extremely well characterised, permitting selection of an age at which the hindlimb muscles manifest a decline in motor function, as well as prominent histopathology. Raman spectra and/or EMG recordings were taken at 90-days of age. A total of 17 mice (n=10 SOD1G93A and n=7 NTg) were used in the study.

For Raman spectra and/or EMG recordings, mice were anaesthetised using 2% isoflurane and placed on a heat pad to maintain body temperature. The hindlimbs were shaved and the optical EMG probe inserted into both gastrocnemius muscles. Raman data were thus obtained from two sites in each mouse (right leg and left leg). For electrophysiological recordings using the optical EMG probe, electrophysiological data were collected from the insertion of the probe into the left medial gastrocnemius. Compound muscle action potentials (CMAPs) were elicited using a 0.1 ms duration stimulus applied at the sciatic notch. Stimulation intensity was adjusted to obtain a supra-maximal response and a baseline-to-negative peak amplitude was recorded. For recordings using the concentric EMG needle probe, recordings were made under anaesthesia from the medial gastrocnemius at the same sitting as the probe recordings, using the same methodology. Mice were humanely sacrificed after recordings.

### Data Analysis

Raman spectral analysis was performed using custom code in MATLAB (MATLAB R2019b The MathWorks). Raw spectra were first interpolated to integer wavenumber spacings between 900 and 1800 cm⁻¹, and subsequently normalised (standard normal variate normalisation) and mean-centred. Spectral windowing between 900 cm⁻¹ and 1800 cm⁻¹ was performed to capture information within the biological fingerprint region. Below this window spectra were obscured by a silica-related Raman signal generated within the optical fibres; above this window spectra consisted only of noise. For the presentation of average spectra, background subtraction using the adaptive, iteratively reweighted penalized least squares (airPLS) algorithm was performed. All SOD1G93A vs. NTg analyses were, however, performed without background subtraction.

For multivariate analyses, principal component fed linear discriminant analysis (PCA-LDA) was performed. For input into the LDA, principal components (PCs) manifesting significant between-group differences (PCs 1 and 2) were used as the inputs into the linear discriminant model. The classification performance of the model was validated using leave-one-mouse-out cross-validation (CV). In this, data from a given mouse were left out and treated as a test set. The models were therefore built using the remaining data. The test set was then projected on the model, performance data collected, and the process was repeated until data from each mouse was left out once, and its group predicted by the model. Accuracy, sensitivity, specificity and area under the receiver operating characteristic curve were reported. Between-group analyses of LDF scores were undertaken using nested (scores nested within each mouse) t-tests on GraphPad Prism (version 9). Differences in CMAP amplitudes between SOD1G93A and NTg mice were analysed using unpaired t-tests; analysis of CMAP amplitudes recorded before and after Raman spectra was performed using paired t-tests.

### Results

To assess both the electrophysiological and Raman spectroscopy functionality of the optical EMG probe testing was undertaken in the SOD1G93A model at 90-days of age. Using the optical EMG probe, CMAPs could be recorded following stimulation of the sciatic nerve (FIG. 2A). In addition, spontaneous EMG activity in the form of positive sharp waves (PSWs) were recorded (FIG. 2B). Significant differences in CMAP amplitudes recorded from SOD1G93A and NTg mice were observed from both the optical EMG probe and the concentric EMG needle (FIG. 2C and FIG. 2D). Thus, high quality, clinically relevant electrophysiological data could be recorded from the optical EMG probe, including waveforms (PSWs) with amplitudes of only 200 μV.

Immediately after the collection of electrophysiological data, Raman spectra were acquired. These comprised peaks associated with muscle and tentative peak assignments were taken from the existing literature. The average spectra demonstrated particularly prominent peaks 935 cm⁻¹ (C-C stretching, protein α-helix), 1000 cm⁻¹ (phenylalanine), 1448 cm⁻¹ (proteins/phospholipids) and 1654 cm⁻¹ (amide I, α-helix) (FIG. 3A). Difference spectra (mean of SOD1G93A minus mean of NTg) demonstrated increased concentrations of peaks relating to protein structure in NTg mice such as 935, 1045, 1448, and 1654 cm⁻¹ (FIG. 3B). Examination of the linear discriminant demonstrated similar peaks to the difference spectrum (FIG. 4A), indicating that these wavenumbers/biochemical components are important for distinguishing between healthy muscle and SOD1G93A muscle pathology. Average LDF scores for each mouse were significantly different at a group level (nested t-test, FIG. 4B). A high classification performance was observed using PCA-LDA (FIG. 4C).

After recording the Raman spectra, CMAP measurements were repeated, keeping the needle of the probe stable in the same location (data represented in FIG. 5A showing SOD1G93A mice and FIG. 5B showing Non-Tg mice). No significant change in CMAP amplitude was seen after Raman spectra were acquired.

### Conclusion

The above demonstrates the functionality of a combined EMG/Raman spectroscopy probe in accordance with the first aspect. The results demonstrate that the probe can record high quality electrophysiological and Raman data in vivo. The data also provide evidence of the utility of optical EMG data as a translational biomarker of muscle health.

As Raman spectroscopy has the potential to provide specific molecular information, the combination of EMG and Raman spectroscopy data is an attractive biomarker for muscle health. On a practical level, concomitant EMG can confirm that the Raman probe is in the muscle of interest. This can be of use in pathological conditions which cause muscle wasting and thus make muscles difficult to palpate. Real time analysis of EMG activity can also be used to target to the Raman probe to electrically abnormal (and normal if desired) areas of muscle, which may increase the likelihood of obtaining molecular information from areas of interest.

The optical EMG probe described herein demonstrated excellent electrophysiological functionality. The method for detecting muscle membrane depolarisation is similar to that of a standard concentric EMG needle. In a standard EMG needle a potential difference is taken between the needle outer wall typically made of steel (acting as a “reference”) and an inner (for example, silver, platinum or any other suitable material such as those described herein) wire called the core (which acts as the active electrode). In the example probe described, the tip of the electrode is ground to an angle of 15 degrees and the close proximity of the needle outer wall and core result in high quality signal due to common mode rejection. Any difference between the electrophysiological functionality of the optical EMG probe (FIG. 6A) and the commercial concentric EMG needle (FIG. 6B) did not result in a statistically significant difference in CMAP amplitudes. Any suitable needle outer wall size may be used and may for example be increased or reduced relative to that described if required. The needle outer wall size in preferable embodiments is greater than or equal to the size described for the embodiment of FIG. 1A and FIG. 1B in order to maximise the ability to collect inelastically scattered (e.g. Raman scattered) light. A smaller diameter needle outer wall may be used in some embodiments, wherein in such embodiments an increased laser power and/or acquisition time may be used to offset for the reduced collection area.

Prominent protein peaks were observed, likely relating to muscle proteins such as myosin and actin. Such Raman spectra can therefore preferably be able to discriminate between neurogenic and myopathic pathology, and different stages of disease. Molecular information, such as that available from Raman spectra obtained using a probe in accordance with the present invention, is not available with any presently available in vivo techniques. Thus, the present probe could provide disease-specific data, which at present is only obtained through muscle biopsy. A preferable key advantage to the present probe would be the potential to examine several areas within a muscle, as well as multiple muscles, as one would typically do in routine EMG. This may increase the likelihood of obtaining disease-specific information, which can sometimes be missed on biopsies of small muscle samples.

No significant difference in post-Raman CMAP amplitudes was observed, indicating that the thermal energy exposure from the laser had not had any deleterious effect on the ability of the muscle fibres to depolarise, highlighting the potential of Raman spectroscopy as a non-destructive technique for tissue analysis.

The present invention therefore provides a technique of EMG/Raman spectroscopic assessment of muscle tissue, combining electrophysiology and vibrational spectroscopy. The data described herein demonstrates that optical EMG can provide sensitive, quantitative measures of disease using the SOD1G93A model of ALS, demonstrating the utility of the present invention for the detection of neuromuscular disease.

FIG. 7A to FIG. 12 are described hereinafter and show results of example training and classification performance of a machine learning module trained using matrix factorisation and applied in accordance with the fifth aspect, with the data suitable for being obtained by a muscle probe in accordance with the first aspect or a system in accordance with the second aspect, the method suitable for providing a digital biomarker in accordance with the sixth aspect. The following description outlines: 1. the methodology used in collecting the data which is represented in FIG. 7A to FIG. 12; 2. the results obtained from the experimentation; and 3. the conclusions drawn from said results.

### Methods

### Fibre Optic Raman Spectroscopy

The fibre optic Raman system utilised a probe in accordance with the first aspect. The system was in accordance with the second aspect. In particular a 0.5 mm probe housed within a 21-gauge hypodermic needle. An 830 nm semiconductor laser was used (Innovative Photonics Solutions), with two low-OH fibres providing the light paths (delivery and collection). In-line bandpass filters were used to remove inelastically scattered light and fluorescence associated with the fibres (Semrock Inc.). A collection fibre was optically coupled to a spectrometer (Raman Explorer Spectrograph, Headwall Photonics, Inc. and iDus 420BR-DD CCD camera, Andor Technology, Ltd.). Laser power at the probe end was 60 mW and the acquisition time for the collection of spectra was 40 seconds for all studies.

### Preclinical Recordings

Breeding was undertaken in a specified pathogen-free environment and experimental work was undertaken in a standard preclinical facility (12-hour light/dark cycle and room temperature 21° C.). As a model of muscle disease, the mdx model of Duchenne muscular dystrophy was utilised at both 30 days (n=16, representing disease onset) and 90 days (n=16, representing a stable phase of disease), with corresponding wild-type healthy control mice (C57BL/10ScSnOlaHsd, n=16 at both 30 days and 90 days). The SOD1^{G93A} model of motor neurone disease was used as a model of ‘neurogenic’ disease (disease relating to nerve or motor neurone pathology) at 90 days (n=16, representing established disease), together with non-transgenic healthy littermate control mice (n=16).

Mice were anaesthetised using 2% isoflurane and hindlimb fur removed. The needle probe was then inserted into the gastrocnemius muscles and the optical fibres were deployed. Two insertions were made in each muscle (the medial and lateral heads of the gastrocnemius muscle) and both legs were studied.

### Human Muscle Sample Recordings

Samples from 54 participants were studied. Briefly, these comprised 10 healthy volunteers with no neurological disease, 17 patients investigated for myopathy but found to have alternative conditions and 27 patients with a final diagnosis of myopathy). Samples were collected either during surgery for a joint injury (healthy volunteers), or via conchotome needle or open biopsy (patients investigated for/diagnosed with muscle disease). Samples were snap frozen and stored at −80° C. until use. For the acquisition of spectra, the fibre optics were pressed gently against the muscle sample. A total of 2-6 sites were studied, depending upon the size of the sample.

### Analysis

Spectral pre-processing, matrix factorisation and classification were undertaken using custom code in MATLAB (R2023a). Peak fitting was undertaken with Origin (2023). Spectra were collected between 900-1800 cm⁻¹, with the lower bound set at 900 cm⁻¹ to avoid the silica-related background from the fibre optics. Spectra were interpolated to even wavenumber spacings and then windowed in the amide I region 1590-1720 cm⁻¹. Averaging (mean) was then performed such that one mouse/human muscle sample presented one spectrum to the subsequent analysis. Background removal was undertaken using the Rubber band algorithm, followed by smoothing (second order Savitzky-Golay filter with a 5 data point window width). Vector normalisation was then performed; as this produces spectra that arbitrarily cross zero, the minimum spectral intensity was added to all spectra to remove the negativity prior to non-negative matrix factorisation.

For peak fitting, group means were generated and scaled (0-1). A mixed Lorentz/Gaussian (Voigt) function was used. For preclinical data, six peaks centered on 1601 and 1615 cm⁻¹ (aromatic amino side chains), 1635 cm⁻¹ (nonregular), 1652 cm⁻¹ (α-helix), 1663 cm⁻¹ (β-sheet) and 1677 cm⁻¹ (nonregular) were used. For human sample analysis, an additional peak at 1705 cm⁻¹ (nonregular) was included. In both preclinical and clinical analyses, the starting height for each peak was the amide I spectral intensity at that wavenumber. Full width at half maximum was enabled to a maximum of 30 cm⁻¹. The percentage of aromatic amino acids and secondary structure components were reported as the percentage of a given peak relative to all peaks utilised in the fitting. Secondary structure ratios were calculated using the percentage integrated area under the peaks of interest (as a proportion of all peaks).

Spectral patterns were derived through a hierarchical alternating least squares non-negative matrix factorisation algorithm optimised for low rank solutions. Briefly, non-negative matrix factorisation approximates the original data (A, an n×m matrix where n is the number of samples and m is the matrix length or number of observations per sample) as the product of two lower rank matrices, A=WH, where W represents the derived non-negative spectral patterns and the matrix H represents the relative importance (the weights, or coefficients) of those patterns to each sample. The number of selected spectral patterns (rank) was determined by calculating the root mean square residual of randomly divided healthy samples in both the preclinical and human datasets, since the difference between two such matrices can be considered to represent biological noise. To estimate the relative contributions of different secondary structures within each mode, the area under the wavenumber region relating to α-helix (1650-1658 cm⁻¹), β-sheet (1664-1673 cm⁻¹) and nonregular (1630-1640, 1674-1689 and 1700-1710 cm⁻¹) was integrated. Secondary structure ratios were calculated using the percentage integrated area under the peaks of interest as a proportion of the total area of that mode. Mode coefficients (weights) were compared using unpaired t-tests (GraphPad Prism, version 9).

For classification, the non-negative factorisation weights were fed into a linear discriminant model and performance statistics derived through a 10-fold cross validation with stratification to ensure balanced classes in the training group (MATLAB). For the three-group mouse analysis, area under the receiver operating characteristic curves (AUROCs) was computed using a one-versus-all approach in which the multiclass classification is reduced to a set of binary classifications. The average AUROC was computed using ‘macro-averaging’ (averaging all the one-versus-all binary results).

### Results

In vivo Raman spectra were collected from the mdx model of Duchenne muscular dystrophy (a form of primary muscle disease) at two disease stages, disease onset and an established disease phase. In vivo spectra were also collected from the SOD1^{G93A} model of ALS (a ‘neurogenic condition arising due to the loss of motor neurone) at an established disease stage. Spectra were also obtained from age-matched non-transgenic/wild type healthy mice (total of 96 mice). Peak fitting of the amide I region was performed to explore secondary protein structure through four mixed Gaussian/Lorentz profiles representing a-helical, β-sheet and non-regular structures (FIG. 7). In addition, two further curves representing aromatic amino acids were included. Detailed peak characteristics are shown in supplemental table one. Of note, full width at half maximum was within the resolution of the system. Utilising the percentage area under each curve, a reduction in the α-helical content in the mdx model was seen (48.3% versus 73.2% for healthy muscle). A smaller reduction in α-helix was observed for SOD1^{G93A} muscle (68%; FIG. 7). There was a corresponding increase in β-sheet content in mdx (19.3% versus 4.6% for healthy muscle and 5.6% for SOD1^{G93A} muscle). Nonregular content was increased in mdx (20.3% versus 10.8% in healthy muscle) and to a lesser degree in SOD1^{G93A} (14.2%). These changes can also be appreciated through α-helix: β-sheet and β-sheet: NR ratios (FIG. 7E).

FIG. 7 shows preclinical peak fitting of healthy, myopathic and neurogenic muscle shows a reduction in alpha helix conformation in myopathy. In particular, FIG. 7A to 7C shows peak fitting using standard peaks within the amide 1 region for healthy, mdx and SOD1^{G93A} mice. FIG. 7D shows each of the peaks resolved as a percentage of the total area. A reduction of α-helix and concomitant increase in β-sheet can be seen in the model of myopathy (mdx). FIG. 7E shows ratios of different protein secondary structure conformations. The reduced α-helix/increased β-sheet is evident in the mdx model of myopathy, which also manifests an increase in the β-sheet/nonregular ratio.

Quantification at the individual sample level (or mouse) was undertaken using non-negative matrix factorisation. As evident in FIG. 8, spectral modes which were dominant in mdx (i.e. mdx had significantly higher scores for that mode), manifested patterns associated with β-sheet and nonregular conformations (e.g. modes 3 and 5). These modes had low α-helix: β-sheet ratios. By contrast, mode 2, which was more dominant in SOD1^{G93A} had no β-sheet region. Feeding these modes into a three-group linear discriminant analysis algorithm demonstrated an average area under the receiver operating characteristic curve of 0.75 (FIG. 9), with the identification of mdx most successful.

FIG. 8 shows non-negative matrix factorisation derived spectral patterns show conformational differences. The unique spectral patterns output from the non-negative matrix factorisation (left) manifest differences in protein structure (middle) and differences between healthy, mdx and SOD1^{G93A} mice (right). The different conformational fingerprints and their predominance in different mice matches those seen via peak fitting (*P<0.05, **P<0.01, ***P<0.001, ****P<0.0001).

FIG. 9 provides a table showing three-group classification performance for in vivo preclinical data using the non-negative matrix factorisation modes and a linear discriminant model.

The matrix factorisation technique employed constrains the outputs to a non-negative distribution. The result is a more interpretable profile than would be obtained through, for example, principal component analysis (PCA). Thus, the spectral modes provide a straightforward shape for integration of the area under the wavenumber windows. This approach was kept intentionally simple, and, although the non-negative constrain often pulls the profile down to zero, it is noted that some area values will be affected by relative importance of adjacent wavenumbers. However, the results align with a simple visual inspection of the modes and their peaks, as well as the more traditional peak fitting data. A preferable benefit of utilising the non-negative approach is that the importance of each spectral mode to each sample allows for quantitation at the level of individual spectra.

It was then assessed if similar conformational fingerprints were evident in human tissue. As patients under investigation for ‘neurogenic’ (e.g. nerve/motor neurone related) conditions such as ALS rarely undergo muscle biopsies, muscle biopsy samples that could be divided into ‘not myopathy’ and ‘myopathy’ groups were analysed (see methods). Peak fitting once again demonstrated a reduced α-helix: β-sheet ratio in the myopathy group and a corresponding decrease in the in the α-helix: NR ratio (FIG. 10). On this occasion an increase in the aromatic amino acid side chains was also evident. These residues cluster in the core of folded proteins and play an important role in the stability of the protein structure. During modifications, such as unfolding, these amino acids become exposed and as such may also represent a marker of muscle health.

FIG. 10 shows peak fitting from Raman muscle spectra from human samples obtained from patients with and without myopathy. In particular, FIG. 10A and B show peak fitting within the amide I region. FIG. 10C shows each of the peaks resolved as a percentage of the total area. A reduction of α-helix and increases in other conformations and aromatic amino acids can be seen in the myopathy group. FIG. 10D shows ratios of different protein conformations. The reduction in α-helix and increased β-sheet/nonregular structures is evident in myopathy.

Non-negative matrix factorisation spectral modes were derived and the mode with the greatest dominance in the ‘not myopathy’ group demonstrated a relatively high α-helix: β-sheet ratio. By contrast, mode 3 which appeared to trend towards dominance in the myopathy group had a low α-helix: β-sheet ratio and a low α-helix: non-regular ratio (FIG. 11). Using these three spectral modes within a linear discriminant classification demonstrated a classification accuracy of 78% (FIG. 12), a result comparable to use of the whole spectrum, with the advantage that the data are more biologically interpretable. The human sample set comprised a range of different human myopathies, which may have differences in protein structure, making for a more challenging classification with the limited number of samples. Human muscle pathology is not homogeneous within a sample (or muscle). Targeting the Raman probe to areas of interest, for example, through concomitant use of electromyography, may also improve detection of disease.

FIG. 11 shows non-negative matrix factorisation derived spectral patterns in human muscle. In particular, the unique spectral patterns output from the non-negative matrix factorisation (left) manifest differences in protein structure (middle). A significant difference was observed for mode 1 (*** P<0.001).

FIG. 12 provides a table showing two-group classification performance for human ex vivo samples using the non-negative matrix factorisation modes and a linear discriminant model.

### Conclusion

The potential of conformational assessments of the Raman amide I region were demonstrated to identify muscle pathology. The approach translates from an in vivo preclinical paradigm to human ex vivo tissue, using a fibre optic system with the potential for in vivo human recording. Through the application of matrix factorisation, it was demonstrated that quantitative information on biologically relevant changes in protein secondary structure can be obtained and used to identify muscle pathology. Conformational fingerprinting is therefore shown as a new, translational biomarker for neuromuscular diseases and, in particular, for myopathy.

FIG. 13 to FIG. 15 are described hereinafter and show results of example training and classification performance of a machine learning module trained using hierarchical modelling and applied in accordance with the fifth aspect, with the data suitable for being obtained by a muscle probe in accordance with the first aspect or a system in accordance with the second aspect, the method suitable for providing a digital biomarker in accordance with the sixth aspect.

### Methods and Results

### Fibre Optic Raman Spectroscopy

A fibre optic Raman system was used in accordance with that for the studies of FIG. 7A to FIG. 12.

### Preclinical Recordings

Breeding was undertaken in a specified pathogen-free environment and experimental work was undertaken in a standard preclinical facility (12-hour light/dark cycle and room temperature 21° C.). As a model of muscle disease, the mdx model of Duchenne muscular dystrophy was utilised at both 30 days (n=16, representing “acute disease”) and 90 days (n=16, representing “chronic disease”), with corresponding wild-type healthy control mice (n=16 at both 30 days and 90 days). The SOD1^{G93A} model of motor neurone disease was used as a model of ‘neurogenic’ disease (disease relating to nerve or motor neurone pathology) at 90 days (n=16). C57BL/10ScSnOlaHsd (C57BI/10) mice at 30 days and 90 days (a background match for the mdx mice) were also used, as were non-transgenic healthy littermate control mice from the SOD1^{G93A} colony. These wild type and non-transgenic mice were then pooled into one “healthy” category.

Mice were anaesthetised using 2% isoflurane and hindlimb fur removed. The needle probe was then inserted into the gastrocnemius muscles and the optical fibres were deployed. Two insertions were made in each muscle (the medial and lateral heads of the gastrocnemius muscle) and both legs were studied.

### Data Analysis

Using custom MATLAB code data were spectra were windowed to 900-1800 cm⁻¹. For all analyses, interpolation to integer wavenumber spacing was undertaken, followed by background subtraction using the adaptive, iteratively reweighted penalised least squares algorithm, Savitzky-Golay smoothing (second order, frame length 5 and standard normal variate normalisation. Outlying spectra (>3 standard deviations from the group mean) were then removed using a rolling window (window length: 20-25 wavenumbers).

Using PLS Toolbox (Eigenvector Research Inc, USA), spectra were then separated into training and test sets using the Kennard Stone algorithm (70% train/30% test). As each mouse may have up to four spectra, this train/test split was done keeping all the data from individual mice either in the train group or the test group (thus data from an individual mouse was not spread across the train/test groups).

The train model comprised n=42 healthy mice (total number of spectra=96), n=17 acute myopathy mice (total number of spectra=39), n=26 chronic myopathy mice (total number of spectra=52), n=20 neurogenic mice (total number of spectra=41). The test model comprised n=17 healthy mice (total number of spectra=60), n=7 acute myopathy mice (total number of spectra=22), n=11 chronic myopathy mice (total number of spectra=33), n=7 neurogenic mice (total number of spectra=19).

Partial least squares discriminant analysis models were then constructed using the train data set for the following two-class problems: healthy vs ‘disease’ (combination of acute myopathy, chronic myopathy and neurogenic groups), myopathy (combination of acute myopathy and chronic myopathy) vs neurogenic and acute myopathy vs chronic myopathy. Prior to the generation of the PLS-DA models, feature selection was undertaken using the variable importance in projection approach and a reduced number of variables selected for inclusion in the PLS-DA models. During construction of the PLS-DA models, cross validation using the venetian blinds method (10 data splits, blind thickness of 1) was used.

Next, a hierarchical classification model was built (as shown schematically in FIG. 13), based upon the clinical decision-making process, taking an expert systems approach. In this hierarchical approach, healthy and disease were first separated. Samples classified as disease were then passed to the next level, at which point samples were classed as either neurogenic or myopathy. Finally, samples selected as myopathy were classified into acute myopathy and chronic myopathy groups.

The train data set samples were then entered into the hierarchical model and classification performance statistics for individual spectra were derived as can be seen in FIG. 14. Performance statistics for the validation test data set are shown in FIG. 15, which show high specificity and accuracy in classifying both sub-types of myopathy—acute and chronic myopathy.

FIG. 16A to FIG. 17B are described hereinafter and show results of example training and classification performance of a machine learning module trained using m modelling and applied in accordance with the fifth aspect, with the data suitable for being obtained by a muscle probe in accordance with the first aspect or a system in accordance with the second aspect, the method suitable for providing a digital biomarker in accordance with the sixth aspect.

### Methods and Results

### Raman Spectroscopy

Raman spectra and CMAP amplitudes were obtained as described herein in relation to FIG. 2A to 6B.

### Multi-Block Modelling of Raman Data in Combination With CMAP Amplitudes

Raman spectral analysis was performed using custom code in MATLAB (MATLAB 15 R2019b The MathWorks). Raw spectra were first interpolated to integer wavenumber spacings between 900 and 1800 cm⁻¹, and subsequently normalised (standard normal variate normalisation). Raman spectra and CMAP amplitudes were then subjected to block variance scaling and then joined to comprise a new data set with 902 variables per mouse (901 spectral wavenumbers and 1 CMAP amplitude). A partial least squares discriminant model was built with venetian blinds cross validation (10 data splits, blind thickness of 1) using 3 latent variables.

The confusion matrix and performance statistics are shown in FIG. 16A and FIG. 16B respectively, which indicates a high sensitivity and specificity in classifying SOD1^{G93A} mice vs non-transgenic mice. These results represent an improvement compared to using the Raman spectra alone (described below and shown in FIG. 17A and FIG. 17B).

### Multi-Block Modelling of Raman Data Alone

Raman spectral analysis was performed using custom code in MATLAB (MATLAB 15 R2019b The MathWorks). Raw spectra were first interpolated to integer wavenumber spacings between 900 and 1800 cm⁻¹, and subsequently normalised (standard normal variate normalisation) and mean centered. A partial least squares discriminant model was built with venetian blinds cross validation (10 data splits, blind thickness of 1) using 1 latent variable.

The confusion matrix and performance statistics are shown in FIG. 17A and FIG. 17B respectively, which indicates a slightly lower sensitivity and specificity in classifying SOD1^{G93A} mice vs non-transgenic mice when compared with modelling both Raman data and CMAP data in combination (as shown in FIG. 16A and FIG. 16B).

The comparative improvement shown in FIG. 16, when CMAP data are combined with data obtained using Raman spectroscopy shows an increase in the classification performance of the model when combining Raman data with CMAP data.

Turning now to FIG. 18, a schematic view of an example embodiment of a system 700 in accordance with the second aspect of the present disclosure is shown. The system 700 comprises a muscle probe 702 substantially as described herein comprising an electromyography needle 704 arranged to be inserted into muscle tissue, the electromyography needle having an outer wall 710 arranged to transmit an electrical signal 712 obtained from the muscle tissue to an electromyography device 714. The needle 704 further comprises a tubular core electrode 716 contained within the outer wall 710, the core arranged to transmit an electrical signal 718 from the muscle tissue to the electromyography device 714. Upon receipt of the electrical signals 712, 718, the electromyography is arranged to output electromyography data 720 characterising the electrophysiological activity of the muscle tissue, for storage in a memory 724 of a processing device 722.

The probe 702 further comprises a plurality of optical fibres 706 encased within the tubular core electrode 716, the plurality of optical fibres 706 comprising a light emitting optical fibre 726 arranged to receive light 728 from a light source 730, and transmit the light 728 toward the muscle tissue. The plurality of optical fibres 706 further comprises three light receiving optical fibres 732 arranged to receive Raman scattered light from the muscle tissue, and transmit the inelastically scattered light 734 to a spectrometer 736. The spectrometer 736 is arranged to generate spectral data from the Raman scattered light 734 and transmit the spectral data 740 to be stored in a memory 724 of the processing device 722. A processor 742 of the processing device is arranged to access the spectral data and the electromyography data 744 from the memory and process the data. Processed data 746 may be transmitted to the memory 742 for storage. It will be appreciated that the system 700 may be used in any manner within the scope as set forth herein. For example, the electromyography data may be obtained prior to obtaining the spectral data, said electromyography data used to inform the positioning of the probe 702 for obtaining said spectral data. As such, the electromyography data may guide the placement of the probe at a desired location for obtaining said spectral data (and optionally further electromyography data) for use in determining a clinical outcome as described herein, or to generate a digital biological fingerprint in accordance with the fourth aspect.

Referring to FIG. 19, a flow chart listing steps of an example embodiment of a method 800 in accordance with the third aspect of the present disclosure is shown. The method comprises: receiving an electrical signal from an electromyography needle, the electrical signal indicative of electrical activity in a muscle 802; determining using the electrical signal, a target muscle location 804; directing a Raman spectroscopy probe to the target muscle location 806; and receiving Raman spectroscopy data from the Raman spectroscopy probe, the Raman spectroscopy data characterising the target muscle location 808. Embodiments will be appreciated wherein spectroscopy data may be obtained in step 802, instead of the electrical signal, and wherein in step 804 the spectroscopy data may be used to determine the target muscle location. Therefore step 806 may instead include directing an electromyography needle to the target location, for instead receiving electromyography data in step 808, characterising the target muscle location.

In the particular example shown, the method further comprises the step of, determining, using the Raman spectroscopy data, and optionally the electrical signal, one or more of: an index of disease state; a prediction of disease state; a predicted disease prognosis; a predicted response to a treatment 810. It will be appreciated that the electric signal may be processed by, for example, an electromyograph to provide a recording of motor unit action potentials and other relevant waveforms, ahead of use said determining step 810. The available processes of performing said determination can include any suitable process, such as by comparing the Raman spectroscopy data, and optionally the electric signal, to stored Raman spectroscopy data, and optionally stored electrical signal data. Other processes may include utilising a machine learning module trained on stored Raman spectroscopy data, and optionally stored electrical signal data, to perform said determining step. Such a determination may, in some embodiments, involve the generation, by the processor, of a digital fingerprint using the Raman spectroscopy data, and optionally the electrical signal (or data obtained therefrom such as Raman spectra or a recording of motor unit action potentials and other relevant waveforms). Such a fingerprint may represent a digital biomarker characterising one or more of: one or more neuromuscular diseases; a prognosis of the muscle and/or associated disease; an index of response of the muscle and/or associated disease to a treatment in accordance with the fourth aspect.

Further embodiments within the scope of the present disclosure may be envisaged that have not been described above, without departing from the scope set out in the appended claims. For example, the particular examples described make use of Raman spectroscopy data. It will be appreciated that the optical fibres may be used to provide any suitable optical spectroscopic assessment (such as fluorescence or Brillouin spectroscopy), the optical fibres permitting a combination of electromyography data and optical spectroscopic data to provide a muscle probe to improve the diagnostic pathway for patients with neuromuscular disorders, constituting a minimally invasive bedside test of muscle health. Additionally, in the particular example described, the muscle probe comprises a tubular electrode housing the optical fibres therewithin. Embodiments will be appreciated wherein the electrode comprises a wire extending within the needle interior alongside the optical fibres. Other embodiments will be appreciated wherein the electrode forms a coating on at least one of the optical fibres. In all embodiments wherein the needle outer wall forms an electrode of the electromyography needle, the active electrode housed therein is electrically insulated therefrom.

1. A muscle probe comprising:
an elongate needle having an outer wall surrounding a needle interior, the needle interior comprising:
a core electromyography electrode; and
one or more optical fibres;
wherein the needle is arranged to be inserted into a muscle, and further arranged to detect electrical activity from the muscle; and
wherein the one or more optical fibres are arranged to direct incident light from a light source toward a target area of the muscle, and further arranged to receive scattered light from the target area.

2. The muscle probe of claim 1, wherein the scattered light comprises inelastic scattered light for assessment using optical spectroscopy.

3. The muscle probe of claim 2, wherein the inelastic scattered light comprises one or more of: Raman scattered light; fluorescence scattered light; Brillouin scattered light.

4. The muscle probe of claim 1, wherein the muscle probe further comprises a cannula, the cannula extending along the needle interior, the core electromyography electrode and/or the one or more optical fibres housed within the cannula.

5. The muscle probe of claim 4, wherein the core electromyography electrode is formed from at least a part of the cannula.

6. The muscle probe of claim 4, wherein the cannula and/or the one or more optical fibres are arranged to move along the needle interior.

7. The muscle probe of claim 1, wherein the core electromyography electrode forms a coating disposed on at least one said optical fibre.

8. The muscle probe of claim 1, wherein the one or more optical fibres comprise:
at least one delivery fibre arranged to direct the incident light from the light source toward the target area of the muscle; and
at least one collection fibre arranged to receive the scattered light from the target area.

9. The muscle probe of claim 8, wherein the one or more optical fibres comprise more collection fibres than delivery fibres.

10. The muscle probe of claim 8, wherein each of the at least one delivery fibre and/or the at least one collection fibre comprises one of: an in-line short-pass filter; an in-line band-pass filter; an in-line long-pass filter; a notch filter.

11. A system for obtaining electromyography data and optical spectroscopy data from muscle, the system comprising:
a muscle probe arranged to be inserted into a muscle, the muscle probe comprising a needle and one or more optical fibres;
a light source arranged to provide incident light for transmission by the one or more optical fibres toward a target area of the muscle;
an optical spectrometer arranged to receive scattered light from the one or more optical fibres; and
an electromyography device arranged to receive an electrical signal from the needle;
wherein the needle comprises an outer wall comprising a needle interior and a core electrode positioned within the needle interior, and wherein the one or more optical fibres are located within the needle interior.

12. The system of claim 11, wherein the one or more optical fibres comprise:
at least one delivery fibre arranged to direct the incident light from the light source toward the target area of the muscle; and
at least one collection fibre arranged to receive the scattered light from the target area.

13. The system of claim 12, wherein each of the at least one delivery fibre and/or the at least one collection fibre comprises one of: an in-line band-pass filter; an inline short-pass filter; an in-line long-pass filter; a notch filter.

14. The system of claim 11, wherein:
the electromyography device is configured to:
determine, using the electrical signal, electromyography data; and
the optical spectrometer is configured to:
determine, using the received scattered light, optical spectra characteristic of the target area.

15. The system of claim 14, further comprising a memory arranged to store the optical spectra and the electromyography data.

16. The system of claim 15, wherein the system further comprises a processor, the processor arranged to perform one or more of:
process the electromyography data and determine, using the electromyography data, the target area; and/or
process the optical spectra, and optionally the electromyography data, and determine using the optical spectra and optionally the electromyography data, a data fingerprint of the target area.

17. The system of claim 16, wherein the processor is further arranged to:
compare the data fingerprint of the target area with one or more stored data fingerprints; and
determine, using said comparison, one or more of: an index of disease state; a prediction of disease state; a predicted disease prognosis; a predicted response to a treatment.

18. The system of claim 16, wherein the processor comprises a machine learning module trained using a plurality of stored fingerprints, the machine learning module arranged to process the data fingerprint and output one or more of:
an index of disease state; a prediction of disease state; a predicted disease prognosis; a predicted response to a treatment.

19. The system of claim 11, wherein the light source is a laser.

20. The system of claim 19, wherein the incident light comprises a wavelength selected from the near infra red spectrum.

21. A computer-implemented method of:
receiving, by the computer, an electrical signal from an electromyography needle, the electrical signal indicative of electrical activity in a muscle;
determining, by the computer, based on the electrical signal, a target muscle location;
outputting, by the computer the target muscle location for directing an optical spectroscopy probe to the target muscle location; and
receiving, by the computer, optical spectroscopy data from the optical spectroscopy probe, the optical spectroscopy data characterising the target muscle location.

22. A computer-implemented method of:
receiving, by the computer, optical spectroscopy data from the optical spectroscopy probe, the optical spectroscopy data characterising a muscle;
determining, by the computer, based on the optical spectroscopy data, a target muscle location;
outputting, by the computer the target muscle location for directing an electromyography needle to the target muscle location; and
receiving, by the computer, an electrical signal from an electromyography needle, the electrical signal indicative of electrical activity in the muscle at the target muscle location.

23. The method of claim 21, further comprising:
determining, by the computer, based on the optical spectroscopy data and optionally further based on the electrical signal, one or more of: an index of disease state; a prediction of disease state; a predicted disease prognosis; a predicted and/or measured response to a treatment.

24. The method of claim 23, wherein said determination is performed by processing the optical spectroscopy data, and optionally the electrical signal, using a machine learning module trained using stored optical spectroscopy data, and optionally stored electrical signals.

25. A method of determining a muscle pathology at a target muscle location of a test subject, the method comprising:
at a computer system comprising at least one processor and a memory storing at least one program for execution by the at least one processor, the at least one program comprising instructions for:
obtaining a dataset, in electronic form, wherein the dataset comprises a test optical spectroscopy data sample obtained from a target muscle location of a test subject; and applying the dataset to a machine learning classifier trained using stored optical spectroscopy data, thereby determining the muscle pathology in the test subject.

26. The method of claim 25, wherein the optical spectroscopy data sample is determined using one or more of: Raman scattering; fluorescence scattering; Brillouin scattering at the target muscle location of the test subject

27. The method of claim 25, wherein the program further comprises instructions for:
isolating, from the test optical spectroscopy data sample, a spectral region comprising spectral data characterising at least one protein secondary structure.

28. The method of claim 27, wherein the spectral region is obtained from the amide I band.

29. The method of claim 27, wherein the spectral data characterises at least a proportion of alpha helix at the target muscle location and a proportion of beta sheet at the target muscle location.

30. The method of claim 25, wherein the target muscle location is determined using an electrical signal from an electromyography needle, the electrical signal indicative of an electrical activity in a muscle.

31. The method of claim 25, wherein the dataset further comprises test electrical signal data from an electromyography needle, the test electrical signal data indicative of electrical activity at the target muscle location of the test subject; and
optionally further wherein the machine learning classifier is further trained using stored electrical signal data.

32. The method of claim 31, wherein the electrical signal data comprises data indicative of one or more of: motor unit action potential at the target muscle location; motor unit action potential morphology at the target muscle location; motor unit action potential configuration at the target muscle location; motor unit action potential recruitment at the target muscle location; spontaneous muscle activity at the target muscle location; compound muscle action potential (CMAP) amplitudes at the target muscle location.

33. The method of claim 25, wherein the machine learning classifier is generated using at least one selected from: matrix factorisation; hierarchical modelling; multi-block modelling; data fusion modelling; principal component analysis.

34. The method of claim 25, wherein the muscle pathology is one selected from: acute muscle myopathy; chronic muscle myopathy; inflammatory myopathy; immune myopathy; dystrophic myopathy; mitochondrial myopathy; inherited myopathy; congenital myopathy; metabolic myopathy; toxic myopathy; endocrine myopathy; infectious myopathy; critical illness myopathy; muscular dystrophy; neurogenic pathology.

35. A digital biomarker determined using either optical spectroscopy data obtained from a muscle, or a combination of optical spectroscopy data and electromyography data obtained from a muscle, the digital biomarker characterising one or more of:
one or more neuromuscular diseases;
a prognosis of the muscle and/or a disease associated therewith;
an index of response of the muscle, and/or a disease associated therewith, to a treatment.

36. The digital biomarker of claim 35, wherein the optical spectroscopy data characterises a proportion of alpha helix and a proportion of beta sheet of said muscle.

37. The digital biomarker of claim 36, wherein the proportion of alpha helix relative to the proportion of beta sheet is below a predetermined threshold.

38. The digital biomarker of claim 37, wherein said predetermined threshold is a proportion of alpha helix relative to a proportion of beta sheet associated with a healthy muscle.

39. The digital biomarker of claim 37, wherein said predetermined threshold is determined by a machine learning module trained on stored optical spectroscopy data obtained from a muscle, or a combination of stored optical spectroscopy data and stored electromyography data obtained from a muscle.

40. The digital biomarker of claim 35, determined using a muscle probe comprising:
an elongate needle having an outer wall surrounding a needle interior, the needle interior comprising:
a core electromyography electrode; and
one or more optical fibres;
wherein the needle is arranged to be inserted into a muscle, and further arranged to detect electrical activity from the muscle; and
wherein the one or more optical fibres are arranged to direct incident light from a light source toward a target area of the muscle, and further arranged to receive scattered light from the target area.

41. A non-transitory computer readable storage medium storing the digital biomarker of claim 35.
